(19) United States
(12) Patent Application Publication
(10) Pub. No.: US 2014/0314869 A1
(43) Pub. Date: Oct. 23, 2014

(54) ISOLATING AND THERAPEUTIC USE OF PERIVASCULAR MEDICINAL CELLS

(71) Applicant: Arnold I. Caplan, Cleveland Heights, OH (US)

(72) Inventor: Arnold I. Caplan, Cleveland Heights, OH (US)

(21) Appl. No.: 14/241,872

(22) PCT Filed: Aug. 27, 2012
(86) PCT No.: PCT/US12/52575

§ 371 Date: Jul. 9, 2014

Related U.S. Application Data
(60) Provisional application No. 61/528,556, filed on Aug. 29, 2011.
(60) Provisional application No. 61/528,563, filed on Aug. 29, 2011.
(60) Provisional application No. 61/528,567, filed on Aug. 29, 2011.

(51) Int. Cl. A61K 35/32; B02C 19/00

(52) U.S. Cl. CPC A61K 35/32; B02C 19/0056
USPC 424/520; 435/325; 435/381; 241/27

(57) ABSTRACT

Disclosed are perivascular medicinal cells (PVMCs), methods of isolating PVMCs, and a composition comprising PVMCs having medicinal capabilities. Specifically, the disclosure provides a method of isolating PVMCs from an umbilical cord blood vessel or from bone. The disclosure further provides a method of making an enhanced, autologous bone graft, a method of stimulating bone regeneration comprising administering a therapeutically effective amount of bone-derived PVMCs, a method of reconstructing bone tissue, and methods of treating a disease that affects cellular function. PVMCs capable of secreting a site-dependent trophic factor are also described.

### CROSS REFERENCE

This application claims the benefit of U.S. provisional patent application Ser. No. 61/528,556 filed on Aug. 29, 2011, U.S. provisional patent application Ser. No. 61/528,563 filed on Aug. 29, 2011, and U.S. provisional patent application Ser. No. 61/528,567 filed on Aug. 29, 2011, each of which is hereby incorporated by reference in its entirety.

### GOVERNMENT INTEREST

Not applicable

### PARTIES TO A JOINT AGREEMENT

Not applicable

### INCORPORATION BY REFERENCE OF MATERIAL SUBMITTED ON A COMPACT DISC

Not applicable

### BACKGROUND

Not applicable

### SUMMARY

Embodiments herein are directed to methods for isolating perivascular medicinal cells (“PVMCs”). Some embodiments are directed to a method for isolating PVMCs from an umbilical cord blood vessel. Some embodiments are directed to a method for isolating PVMCs from an umbilical cord blood vessel, comprising draining the umbilical cord blood vessel; adding a first enzyme mixture to the umbilical cord blood vessel to disassociate the PVMC; adding a medium; and collecting a wash eluent after adding the medium, wherein the wash eluent comprises a cell suspension of cells selected from endothelial, subendothelial cells, and combinations thereof. In some embodiments, the umbilical cord blood vessel is a vein or artery.

In some embodiments, the first enzymatic mixture comprises an enzyme selected from collagenases, neutral or acidic proteases, GAGases, metalloproteases clostripain, serine proteases, alkaline proteases, cysteine proteases, and combinations thereof. In some embodiments, the first enzyme mixture further comprises a second agent selected from a medium, an antibiotic, and a combination thereof. In some embodiments, the medium is selected from Tyrode's solution, lactated Ringer's Solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), and combinations thereof. In some embodiments, the antibiotic is selected from streptomycin, gentamicin, fungizone, penicillinG, amphotericin B, and combinations thereof.

Some embodiments of the method further comprise incubating the umbilical cord blood vessel with the first enzyme mixture at a temperature ranging from about 15° C. to about 38° C. In some embodiments, the umbilical cord blood vessel may be incubated with the first enzyme mixture for about 15 to about 60 minutes. In some embodiments, the method further comprises inactivating the enzyme.

In some embodiments of the method, the medium is selected from Tyrode's solution, lactated Ringer's Solution, acetated Ringer's solution, TRIS-buffered saline (TBS), Hank's balanced salt solution (HESS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof.

Some embodiments of the method further comprise adding a second enzyme mixture to the umbilical cord blood vessel. In some embodiments, the second enzyme mixture comprises an enzyme selected from collagenases, neutral or acidic proteases, GAGases, metalloproteases clostripain, serine proteases, alkaline proteases, cysteine proteases, or a combination thereof. In some embodiments, the second enzyme mixture further comprises a second agent selected from a medium, an antibiotic, and a combination thereof. In some embodiments, the medium is selected from Tyrode's solution, lactated Ringer's Solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof. In some embodiments, the antibiotic is selected from streptomycin, gentamicin, fungizone, penicillinG, amphotericin B, or a combination thereof.

Some embodiments of the method further comprise incubating the umbilical cord blood vessel with the second enzyme mixture at a temperature ranging from about 15° C. to about 38° C. In some embodiments, the umbilical cord blood vessel is incubated with the second enzyme mixture for about 15 to about 60 minutes. In some embodiments, the method further comprises inactivating the enzyme.

Some embodiments of the method further comprise adding a second medium to the umbilical cord blood vessel after incubating the umbilical cord blood vessel with the second enzyme mixture. In some embodiments, the second medium is selected from Tyrode's solution, lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof.

Some embodiments of the method further comprise collecting a second wash eluent after adding the medium. In some embodiments, the second wash eluent comprises a cell suspension of cells selected from endothelial, subendothelial cells, and combinations thereof.

Some embodiments of the method further comprise washing the second eluent with a medium. In some embodiments, the medium may be selected from Tyrode's solution, lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof. In some embodiments of the method, the cell suspension may comprise PVMCs.

Some embodiments of the method further comprise culturing the cell suspension in a cell culture medium. In some embodiments, the cell suspension may comprise PVMCs and wherein the PVMCs are capable of adhering to a cell culture dish. Some embodiments of the method further comprise concentrating the PVMCs to yield a population of concentrated disaggregated PVMCs.

Some embodiments of the method further comprise isolating the PVMCs from the cell suspension cultured in a cell culture medium.

Some embodiments are directed to a method for isolating PVMCs from bone. Some embodiments are directed to a method for isolating PVMCs from bone, the method comprising: (i) providing a sample of bone tissue from a subject; (ii) extracting the PVMCs from the bone; and (iii) concentrating the extracted PVMCs. In some embodiments, extracting the PVMCs comprises: (i) extracting a cell suspension from the bone tissue by enzymatic digestion, mechanical force, or a combination thereof; and (ii) separating a population of PVMCs from the cell suspension by buoyant density sedimentation, filtration, centrifugation, or a combination thereof. Some embodiments further comprise grinding the bone tissue. In some embodiments, the enzymatic digestion uses one or more enzymes that cleave the attachment of a PVMC from a basement membrane of a small blood vessel. In some embodiments, concentrating the extracted PVMCs is achieved by methods comprising the use of magnetic beads containing antibodies with affinity to cell surface antigens on the PVMC. In some embodiments, the antibodies are selected from anti-CD146, anti-CD105, anti-CD166, anti-CD271, or a combination thereof.

Some embodiments are directed to compositions comprising PVMCs. Some embodiments are a composition comprising a plurality of PVMCs and an acceptable carrier. In some embodiments of the composition, the plurality of PVMCs comprises PVMCs derived from bone, an umbilical cord blood vessel, or a combination thereof. In some embodiments of the composition, the bone comprises bone chips, bone marrow tissue and other tissue, compact bone, bone marrow from an intermedullary canal, a bone chip, a trabecular bone cavity, a bone cavity lavage, or combinations thereof. Some embodiments of the composition further comprise bone marrow cells. Some embodiments of the composition further comprise a scaffold material. In some embodiments of the composition, the scaffold material comprises bone chips, ceramic-based bone graft substitutes, calcium phosphate ceramics, calcium sulfate ceramics, bioglass, polymer-based bone graft substitutes, degradable and nondegradable polymers, processed allograft bone material, mineralized processed allograft, demineralized processed allograft, collagen sponges, or combinations thereof.

Some embodiments are directed to a PVMC. In some embodiments, the PVMC is derived from bone. In some embodiments, the PVMC is derived form an umbilical cord blood vessel. In some embodiments, the bone comprises a bone chip, a trabecular bone cavity, bone marrow, bone cavity lavage, or a combination thereof.

Some embodiments are directed to pharmaceutical compositions comprising PVMCs. Some embodiments are directed to a pharmaceutical composition comprising a therapeutically effective amount of a plurality of isolated PVMCs and a pharmaceutically acceptable carrier. In some embodiments, the plurality of PVMCs comprises PVMCs derived from bone, umbilical cord blood vessel, an anatomic source containing PVMCs, or a combination thereof. In some embodiments, the bone comprises a bone chip, a trabecular bone cavity, bone marrow, bone cavity lavage, or a combination thereof. Some embodiments of the pharmaceutical composition further comprise bone marrow cells. Some embodiments of the pharmaceutical composition further comprise a scaffold material. In some embodiments, the scaffold material comprises bone chips, ceramic-based bone graft substitutes, calcium phosphate ceramics, calcium sulfate ceramics, bioglass, polymer-based bone graft substitutes, degradable and nondegradable polymers, processed allograft bone material, mineralized processed allograft, demineralized processed allograft, collagen sponges, or combinations thereof.

Some embodiments are directed to a method of making an enhanced, autologous bone graft comprising (i) extracting from a subject a first portion of bone tissue to be used as a bone graft, then (ii) supplementing the bone graft with a population of concentrated PVMCs.

Some embodiments are directed to a method for making an enhanced, autologous bone graft comprising:
a. extracting a cell suspension from a first portion of bone tissue from a subject with an enzyme, mechanical force, or a combination thereof;
b. concentrating the cells in the cell suspension by buoyant density sedimentation, filtration or centrifugation to obtain a population of concentrated bone-derived PVMCs; and
c. supplementing a second portion of bone tissue to be used as a bone graft from the subject with the population of concentrated bone-derived PVMCs, so as to make the enhanced, autologous bone graft.

In some embodiments, the first portion of bone tissue originates from the proximal region of a femur, the distal region of a femur or a combination thereof. In some embodiments, the second portion of bone tissue originates from human bones comprising at least one of an ilium crest, a femur, a patella, a tibia, a humerus, a clavicle, a rib, a scapula, or a combination thereof. Some embodiments further comprise supplementing the enhanced, autologous bone graft with fresh autologous bone marrow, processed autologous bone marrow, frozen autologous bone marrow, fresh autologous bone, processed autologous bone, frozen autologous bone, or a combination thereof.

Some embodiments are directed to a method of treating a disease that affects cellular function comprising administering a composition comprising a therapeutically effective amount of PVMCs to a subject in need thereof. In some embodiments, the PVMCs are capable of secreting a site-dependent trophic factor. In some embodiments, the site-dependent trophic factor is selected from prostaglandin E2 (PGE2), stromal-cell derived factor-1 (SDF-1 Vascular endothelial growth factor (VEGF), VEGF165, interleukin-1β (IL-β), interleukin-6 (IL-6), interleukin-7 (IL-7), interleukin-8 (IL-8), interleukin-12 (IL-12), interleukin-16 (IL-16), hepatocyte growth factor (HGF), transforming growth factor beta (TGF-β), basic fibroblast growth factor (bFGF), granulocyte-macrophage colony-stimulating factor (GM-CSF), insulin-like growth factor 1 (IGF-1), indoleamine 2,3-dioxygenase (IDO), interleukin-10 (IL-10), human leukocyte antigen G (HLA-G), leukemia inhibitory factor (LIF), class II major histocompatibility complex (MHC), eotaxin, granulocyte colony stimulating factor (G-CSF), regulated upon activation, normal T-cell expressed and secreted (RANTES), IL-1 receptor antagonist (IL-1ra), tumor necrosis factor-α, TNF-α, tumor necrosis factor-β (TNF-β), epithelial neutrophil-activating protein 78 (ENA-78), eotaxin, monocyte chemoattractant protein 1 (MCP-1), monocyte chemoattractant protein 3 (MCP-3), macrophage inflammatory protein-1α (MIP-1α), macrophage inflammatory protein-3 α (MIP-3 α), macrophage inflammatory protein-1 β (MIP-1 β), intercellular adhesion molecule-1 (ICAM-1), VCAM-1, granulocyte colony-stimulating factor (G-CSF), growth hormone, stem cell factor (SCF), thyroid-stimulating hormone (TSH), CD40 and CD40 ligand, placental growth factor (PlGF), eotaxin-3, fractalkine, epithelial neutrophil-activating protein 78 (ENA-78), Interferon-inducible T-cell alpha chemoattractant (i-TAC), growth regulated oncogene-alpha (GROα), growth regulated oncogene-beta (GROβ), Interferon-inducible protein-10 (IP-10), CD146, CD105, CD166, CD44, CD271, CD73, CD90, CD10, or a combination thereof. In some embodiments, the disease is ischemic heart disease, burns, stroke, inflammatory bowel disease, Crohn's disease, rheumatoid arthritis, lupus, amyotrophic lateral sclerosis, spinal cord damage, polytrauma, bone fractures, diabetes, or combinations thereof.

Some embodiments are directed to a method of reconstructing bone tissue comprising administering a composition comprising a therapeutically effective amount of PVMCs to a subject in need thereof.

Some embodiments are directed to a method of anchoring a metal device within a bone comprising administering a composition comprising a therapeutically effective amount of PVMCs to a subject in need thereof. In some embodiments, the metal device is anchored in a bone selected from cranial-facial bone, cranium, mandible, clavicle, scapula, sternum, ribs, humerus, ulna, radius, carpels, phalange, metacarpal, patella, fibula, femur, tibia, tarsal, metatarsal, sacrum, coxa or lumbar vertebrae.

Some embodiments are directed to a method of modulating apoptosis comprising administering a composition comprising a therapeutically effective amount of PVMCs to a subject in need thereof.

Some embodiments are directed to a method of modulating mitosis comprising administering a composition comprising a therapeutically effective amount of PVMCs to a subject in need thereof.

Some embodiments are directed to a method of modulating angiogenesis comprising administering a composition comprising a therapeutically effective amount of PVMCs to a subject in need thereof.

Some embodiments are directed to a method of modulating bone formation comprising administering a composition comprising a therapeutically effective amount of PVMCs to a subject in need thereof. In some embodiments, the perivascular medicinal cells have the capability to form osteoblasts.

Some embodiments are directed to a method of immunomodulation comprising administering a composition comprising a therapeutically effective amount of PVMCs to a subject in need thereof.

Some embodiments are directed to a method of producing bone chips comprising passing a bone fragment through a grinder or bone mill. In some embodiments, the bone fragment is cryogenically frozen.

Some embodiments are directed to a method of separating osteogenic cells from a PVMC preparation comprising determining adsorption of a cell in the preparation to calcium phosphate substrates, wherein a high affinity indicates the presence of an osteogenic cell.

### DESCRIPTION OF THE DRAWING

FIG. 1 is a flow chart depicting the exemplary steps for isolating PVMCs from an umbilical cord blood vessel according to an embodiment described herein.

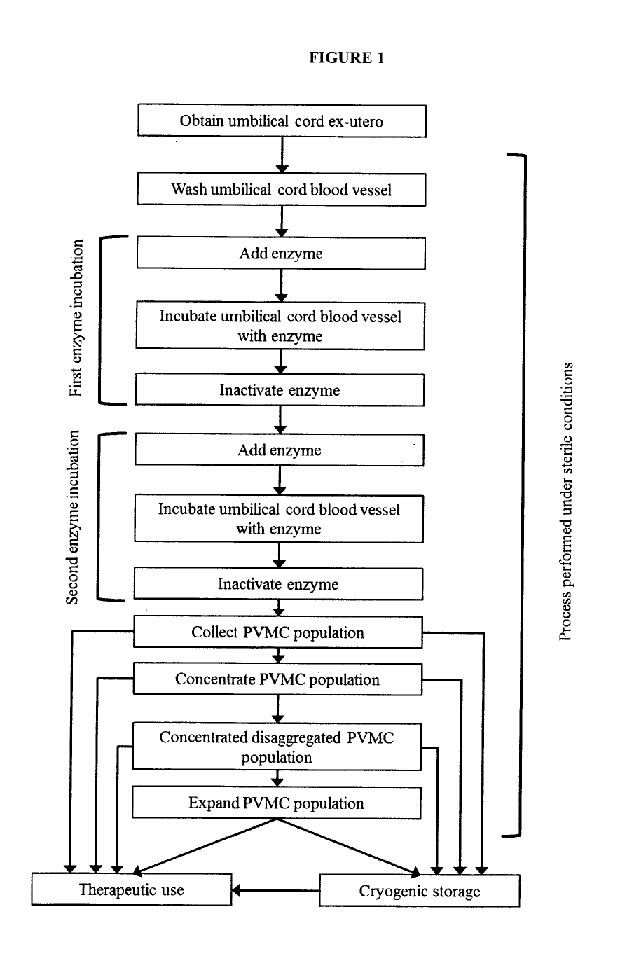

### DETAILED DESCRIPTION

Some embodiments generally relate to cells, human tissue, and more particularly, to human tissue-derived PVMCs, methods of using human tissue-derived PVMCs, compositions containing bone-derived and umbilical cord blood vessel-derived PVMCs, and systems for preparing and using bone-derived and umbilical cord blood vessel-derived PVMCs.

The identification of mesenchymal stem cells (MSCs) as pericytes has given new meaning to the process of bone formation, regeneration, and repair. Growth factors and signaling molecules together with MSCs play an important role in these processes.

The formation of new vasculature also plays a crucial role in bone growth, regeneration, and repair, both in driving the process and orienting bone formation. Signaling molecules such as platelet derived growth factor (PDGF) are believed to function in stimulating osteoblast differentiation into bone. Physiologically, PDGFs may recruit pericytes to the site of injury from their abluminal dwelling, trigger an expansion of the cell population, and control the growth and differentiation of osteoblasts, as well as promote new vessel formation.

There is a need for alternate approaches in which a population of active PVMCs with increased yield, consistency, and/or purity can be prepared rapidly and reliably, and whereby the need for post-extraction manipulation of the cells can be reduced or eliminated. Ideally, this cell population would be obtained in a manner that is suitable for their direct placement into a recipient.

In vitro, pericytes, or perivascular cells are multipotent for osteogenic, chondrogenic, adipogenic and myogenic lineages and are similar to MSCs in their cell surface expression profile (CD146+, CD34−, CD45−, and CD46−).

Localized perivascular cells may play a role in physiological bone healing (i.e. callus formation). Furthermore, the endochondral replacement of cartilage by the vasculature brings perivascular cells to the site of injury. These perivascular cells may be capable of differentiating in vascular-driven bone in both orthotopic and heterotopic locations.

A bone injury, such as, without limitation, a broken bone, may be characterized by a separation between two pieces of bone that were formerly joined. The gap that is created between the two pieces of bone fills with mesenchymal progenitor cells that differentiate into cartilage (a mechanically unstable break) or allow blood vessels to span the break (mechanically stable break). These space filling cells span the gap and provide a connection between the broken edges forming connective tissue. The drivers of bone formation following bone injury through the connecting space and outside the break are blood vessels which orient the progenitor cells to become bone forming osteoblasts that are oriented with their basolateral side facing the blood vessel and coordinately from their apical sides they secrete osteoid which eventually becomes mineralized to form weight-bearing bone. In some embodiments, the PVMCs of embodiments herein may be used to fulfill the role of mesenchymal progenitor cells.

Autologous bone grafting may be an effective tool to induce osteogenic regeneration following, for example but without limitation, bone injury, where local bone defects exist, and in pseudoarthroses. Without wishing to be bound by theory, it is believed that autologous bone grafting with bone marrow aspiration concentrates (BMAC) may contain mesenchymal stem cells, which may play a part in regulating immune cell proliferation, differentiation and phenotype, attenuate inflammation and injury, and produce effector molecules that drive tissue regeneration.

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “perivascular medicinal cell” (PVMC) is a reference to one or more PVMCs and equivalents thereof known to those skilled in the art and so forth.

As used herein, the term “about” means plus or minus 10% of the numerical value of the number with which it is being used. Therefore, about 50% means in the range of 45%-55%.

The term “perivascular medicinal cell” or “PVMC”, as used herein, refers to mononuclear cells, endothelial cells, subendothelial cells, perivascular cells, or osteogenic cells. A PVMC mononuclear cell is characterized by the expression of one, some or all of the cluster of differentiation (CD) markers selected from CD146+, CD271+, CD90+, CD166+, CD73+, CD105+, CD44+, CD29+, SSEA4+, CD45−, CD31−, vWF−, and CD14−, or a combination thereof. A PVMC endothelial or subendothelial cell is characterized by the expression of one, some, or all of Syto16+, CD45−, CD31+, CD156+, or a combination thereof. A PVMC osteogenic cell is characterized by the expression of one, some, or all of alkaline phosphatase, osteopontin, osteocalcin, or a combination thereof. PVMCs may be negative for MHC class I but may express MHC class II. Isolated PVMCs may be distinguished from other cell types on the basis of presence of markers, such as cell surface polypeptides. Detection of these markers may be performed using immunocytochemistry, fluorescence-activated cell sorting (FACS), reverse transcription polymerase chain reaction (RT-PCR) or the like. Useful markers for identifying PVMCs may include, without limitation, Growth Factor Receptors: CD121 (IL-1R), CD25 (IL-2R), CD123 (IL-3R), CD71 (Transferrin receptor), CDI17 (SCF-R), CD114 (3-CSF-R), PDGF-R and EGF-R; Hematopoietic markers: CD1a, CD11b, CD14, CD34, CD45, CD133; Adhesion receptors: CD166 (ALCAM), CD54 (ICAM-1), CD102 (ICAM-2), CD50 (ICAM-3), CD62L (L-selectin), CD62e (E-selectin), CD3I (PECAM), CD44 (hyaluronate receptor); Integrins: CD49a (VLAα), CD49b (VLA α2), CD49c (VLA-α3), CD49d (VLA-α4), CD49e (VLA α5), CD29 (VLA-β), CD 104 (β4-integrin); and other miscellaneous markers: D90 (Thy1), CD105 (Endoglin), CD80 (B7-1) and CD8 (B7-2). In some embodiments, PVMCs may include cells derived from an umbilical cord blood vessel which express CD146+, CD271+, CD90+, CD166+, CD73+, CD105+, CD44+, CD29+, SSEA4+, CD45−, CD31−, vWF−, CD14−, or a combination thereof. In some embodiments, PVMCs may include cells derived from an umbilical cord blood vessel, endothelial cells, osteogenic cells, or a combination thereof. In some embodiments, PVMCs may be derived from any anatomic source that may contain PVMCs. Examples of anatomic sources include, but are not limited to, blood vessels including but not limited to veins and arteries, periosteum, trabecular bone, adipose tissue, synovium, skeletal muscle, deciduous teeth, pancreas, lung, liver, amniotic fluid, placenta and blood. PVMCs may express, without limitation, cell surface markers CD29, CD105, CD44, CD73 CD146 and CD166. In some embodiments, PVMC preparations may be over 90% pure in terms of antigen expression and viability and to express a phenotype of CD73+/CD105+/CD44+/CD29+/SSEA4+/CD45−/CD31−/vWF−/CD14−. These PVMCs may be negative for MHC class I but expressed MHC class II. In some embodiments, PVMC preparations are expected to be less than 90% pure, less than 80% pure, less than 70% pure, less than 60% pure, or less than 50% pure.

PVMC cell surface markers may be characterized by FACS after being labeled with various antibodies, including those against human CD29, CD105, CD44, CD73, SSEA4, CD45, CD31, vWF, and CD14. In some embodiments, secondary antibodies conjugated with fluorescin may be subsequently used.

As used herein, the term “isolated PVMC” refers to a PVMC, PVMC population or PVMC preparation wherein the PVMCs have been isolated from an organism. In some embodiments, the organism is a mammal. In some embodiments, the mammal is a gestational mammal. In some embodiments, the gestational mammal is a human. In some embodiments, an isolated PVMC is a PVMC isolated from periosteum, trabecular bone, adipose tissue, synovium, skeletal muscle, deciduous teeth, pancreas, lung, liver, amniotic fluid, placenta, blood and umbilical cord blood vessel, or combinations thereof. In some embodiments, an isolated PVMC is a PVMC derived from bone. In some embodiments, an isolated PVMC is a PVMC derived from an umbilical cord blood vessel.

In some embodiments, a PVMC complex may be isolated. In some embodiments, a PVMC complex comprises a group of cells. In some embodiments, the PVMC complex may include an osteogenic cell, a mononuclear cell, an endothelial cell a subendothelial cell, mesenchymal stem cell, or a combination thereof. In embodiments where an autologous graft is used or made, the PVMC may be a PVMC complex or a purified PVMC.

PVMCs exhibit extensive diversity in differentiation, production of trophic mediators, and interaction with the host environment. In some embodiments, the PVMCs of the present invention may be autologous, allogeneic, or from xenogeneic sources. In some embodiments, the PVMC are intended for autologous use. PVMCs can be isolated and these cells can be administered back to the patient from whom they were raised. This technique of autologous transfer prevents the need for immunosuppressive protocols. In some embodiments, PVMCs can be embryonic or from post-natal sources. Bone marrow may be obtained from iliac crest, femora, tibiae, spine, rib or other medullary spaces, or combinations thereof. Other sources of PVMCs include fat, periosteum, skin, and skeletal muscle, liver, placenta, blood and umbilical cord blood vessels, or combinations thereof.

As used herein, the term “medicinal capabilities” refers to the spectrum of molecules secreted by the PVMC in a particular physiological environment. PVMCs derive their medicinal properties from the vast array of bioactive molecules that contribute to immunomodulatory functions and separately offer so-called “trophic effects” by providing a regenerative environment at the site of injury.

As used herein, the term “trophic effects” refers to the spectrum of molecules secreted by the PVMC in a site dependent fashion. In some embodiments, trophic effects of PVMCs will vary depending on the physical location of the PVMC. For example, in some embodiments, secretion and fabrication of bioactive molecules by PVMCs may result in T-cell inhibition by affecting antigen presentation and T-cell progenitor expansion, protecting the injury site from immune surveillance and forestall autoimmunity sensitization to the damaged tissue. In some embodiments, PVMCs may have anti-apoptotic effects in ischemic tissue. Molecules secreted by PVMCs may be able to protect against cell death resulting from broken or malfunctioning blood vessels that do not permit normal levels of oxygen or nutrients to enter an injured tissue. In some embodiments, PVMCs may have anti-scarring or anti-fibrotic effects. Molecules secreted by PVMCs may inhibit the entrance or function of myofibroblasts in a wound site, resulting in inhibition of the formation of dense collagenase scar-tissue. In some embodiments, PVMCs may have angiogenic effects. Molecules secreted by PVMCs may be able to elicit recruitment of endothelial cells or their progenitors into an injury site where they can divide and form primitive blood vessels. In some embodiments, the PVMC may itself develop into a pericyte, attaching to the newly formed blood vessel and providing stability to the nascent vessels. In some embodiments, PVMCs may secrete mitogens that affect tissue intrinsic progenitors to divide and differentiate and regenerate tissue at a site of injury. In some embodiments, PVMCs may secrete powerful chemoattractants capable of recruiting a variety of repair and helper cells into a site of injury and promote tissue regeneration.

PVMCs may be capable of migrating to the site of injury in response to digested extracellular matrix (ECM) as well as other chemotactic stimuli. Secretion or fabrication of bioactive molecules by PVMCs may result in T-cell inhibition by affecting antigen presentation and T-cell progenitor expansion, protecting the injury site from immune surveillance and forestall autoimmunity sensitization to the damaged tissue.

Administration of PVMCs to a subject in need thereof results in an augmentation of the physiological and therapeutic effect of a PVMC due to an increased number of PVMCs present at a site of injury. In some embodiments, administration of PVMCs to a subject in need thereof results in an increase in the number of PVMCs within the subject. In some embodiments, the physiological and therapeutic effect of PVMC administration is positively correlated with the number of PVMCs present at the site of injury.

As used herein, the term “cell medium” or “cell media” is used to describe a cellular growth medium in which mononuclear cells and/or neural cells are grown. Cellular media are well known in the art and comprise at least a minimum of essential medium plus optional agents such as growth factors, glucose, non-essential amino acids, insulin, transferrin and other agents well known in the art.

As used herein, the term “non-adherent cells” is used to describe cells remaining in suspension in the tissue culture flask at the end of the culture period.

The term “adherent cells” is used to describe cells that are attached to the tissue culture plastic and are detached from the flask by addition of enzyme-free cell dissociation buffer from Gibco-BRL or by addition of trypsin-EDTA.

As used herein, the term “mononuclear cells” is used to describe cells containing a single nucleus isolated from bone marrow or umbilical cord blood vessels or blood. Mononuclear cells may be isolated using a density gradient of FICOLL™ or PERCOLL™. Mononuclear cells are obtained from bone marrow or umbilical cord blood vessels or blood and are used as a source of PVMCs.

As used herein, the term “bone” refers to bone marrow tissue and other bone-related tissue, compact bone including, without limitation, bone chips, bone fragments, bone powder, bone segments or the like, bone marrow from an intermedullary canal, or combinations thereof. In some embodiments, the term “bone” includes bone marrow.

As used herein, the term “bone derived” refers to material isolated from, without limitation, a bone, bone chip, bone powder, bone segment, bone fragment, bone marrow or bone cavity lavage. Bone cavity lavage is performed following physical removal of bone marrow from a bone cavity.

As used herein, the term “bone tissue” refers to tissue from, without limitation, a bone, bone chip, bone powder, bone segment, bone fragment, bone marrow scoop or bone cavity lavage.

The term “bone marrow cells” refers to fibroblasts (reticular connective tissue), macrophages, adipocytes, osteoblasts, osteoclasts, endothelial cells forming the sinusoids, hematopoietic stem cells, mesenchymal stem cells, endothelial stem cells, pericytes, PVMCs, tissue helper cells, or combinations thereof.

As used herein, the term “disassociate” refers to the process of releasing PVMCs from the basement membrane surrounding a blood vessel such that they may be separated from the blood vessel tissue. In some embodiments, the blood vessel is an umbilical cord blood vessel. Disassociation of a PVMC is achieved by enzymatic digestion of the bonds joining the PVMCs to a basement membrane of small blood vessels in the umbilical cord. In some embodiments, enzymatic digestion cleaves bonds of the basement membrane that house separated, associated molecules to which the PVMCs separately associate. For example, PDGF-BB, which binds to heparin in the basement membrane, and PDGF-BB, in turn, may be capable of binding to PVMCs which express the PDGF receptor. In some embodiments, the enzymatic digestion may be achieved by using one or more enzymes selected from: collagenases, neutral or acidic proteases, GAGases, or metalloproteases, clostripain, serine proteases, alkaline proteases, cysteine proteases, or combinations thereof. The one or more enzyme may be, without limitation, from an animal, plant, bacteria, or fungi, or a combination thereof. In some embodiments, the enzymatic digestion uses one or more enzymes that cleave the attachment of a PVMC from a basement membrane of a small blood vessel. In some embodiments, an enzymatic digestion also includes enzymatic digestion to cleave bonds joining the PVMC to molecules that may be themselves bound to the basement membrane. For example, PDGF-BB which binds to heparin in the basement membrane, and PDGF-BB in turn may be capable of binding to PVMCs which express the PDGF receptor.

As used herein, the term “flush” refers to the process of filling and emptying a cavity such as a clamped blood vessel with a liquid. In some embodiments, the process of filling is immediately followed by emptying. In some embodiments, the process of filling and emptying is separated by an incubation period. In some embodiments, the liquid is retained for further use. In some embodiments, the liquid is discarded.

Methods for Isolating PVMCs from an Umbilical Cord Blood Vessel

In some embodiments, PVMCs may be isolated from periosteum, trabecular bone, adipose tissue, synovium, skeletal muscle, deciduous teeth, pancreas, lung, liver, amniotic fluid, placenta, blood and umbilical cord blood vessel. In some embodiments, umbilical cord blood vessel blood and umbilical cord blood vessels may be particularly advantageous sources of PVMCs because of their availability, non-invasiveness, and potential for autologous cell-based therapy. The umbilical cord blood vessel contains two arteries and one vein surrounded by mucoid connective tissue known as Wharton's jelly. In some embodiments, PVMCs may be isolated from perivascular regions of the umbilical cord arteries and vein.

The umbilical cord blood vessel in a full term neonate may be about 50 centimeters long and about 2 centimeters in diameter. In some embodiments, the umbilical cord blood vessel of embodiments herein may have a length of about 25 cm to about 60 cm, from about 30 to about 60 cm, from about 35 to about 60 cm, from about 40 to about 60 cm, from about 45 to about 60 cm, from about 30 to about 55 cm, from about 35 to about 55 cm, from about 40 to about 55 cm, from about 45 to about 55 cm, from about 50 to about 55 cm, about 40 cm, about 45 cm, about 50 cm, about 55 cm, or a range between any two of these values.

In some embodiments, isolating umbilical cord blood vessel blood from full term deliveries can be performed ex utero from the freshly delivered placenta, following full term normal delivery or caesarian section. In some embodiments, isolating umbilical cord blood vessel blood may comprise suspending the placenta, cannulating the vein and allowing the blood to drain by gravity into a specially designed collection bag or container. Though there is no risk to the mother or infant during ex utero isolating, the risk of microbial contamination of the umbilical cord blood vessel blood is high.

In some embodiments, a method of isolating PVMCs from an umbilical cord blood vessel comprises adding an enzyme or enzyme mixture to the umbilical cord blood vessel to dissociate the PVMCs from the umbilical cord arteries or vein. Some embodiments further comprise draining the umbilical cord vessel before adding the enzyme or enzyme mixture. In some embodiments, the isolating process is sterile. In some embodiments, the umbilical cord blood vessel may be isolated from any placental mammal. In some embodiments, the umbilical cord blood vessel may be human or from a non-human placental mammal such as, without limitation, a wild, domesticated, or farm animal.

In some embodiments, the method of isolating PVMCs from an umbilical cord blood vessel comprises (i) draining the content of an umbilical cord blood vessel and inserting a needle into the umbilical cord blood vessel, (ii) flushing the umbilical cord blood vessel with sterile phosphate buffered saline, and (iii) collecting the content. In some embodiments, the process of draining the contents of the umbilical cord blood vessel comprises inserting two needles with stoppers, one in a top portion and another in a bottom portion of an umbilical cord blood vessel, emptying the blood vessel and collecting the wash eluent. In some embodiments, the blood vessel may be a vein or an artery. In some embodiments, emptying the blood vessel comprises allowing gravity to empty the blood vessel.

In some embodiments, an enzyme mixture is incubated within the umbilical cord blood vessel. In some embodiments, the enzyme mixture is incubated within the umbilical cord blood vessel after draining, flushing and collecting the content of the umbilical cord blood vessel. In some embodiments, the enzyme mixture comprises collagenases, neutral or acidic proteases, GAGases, metalloproteases clostripain, serine proteases, alkaline proteases, cysteine proteases, or combinations thereof. In some embodiments, the enzymes may be, without limitation, from an animal, plant, bacteria, or fungi, or a combination thereof. Collagenases are enzymes that break the peptide bonds in collagen. Collagen is the main component of connective tissue and is the most abundant protein in mammals and is also a key component of the animal extracellular matrix. Proteases, also known as proteolytic enzymes, are capable of performing protein catabolism by hydrolysis of the peptide bonds that link amino acids together in the polypeptide chain forming the protein. Proteases comprise a number of broad groups of enzymes including serine proteases, threonine proteases, cysteine proteases, aspartate proteases and glutamic acid proteases. Proteases are further classified by the optimal pH at which they function best. Some proteases can break specific peptide bonds in proteins while others are capable of complete digestion of a protein to individual amino acids. In addition to mammalian proteases, a number of bacterial, fungal and plant proteases also exist. Proteases are capable of digesting long protein chains into short fragments by splitting the peptide bonds that link amino acid residues. Some proteases can detach the terminal amino acids from the protein chain (exopeptidases, such as amino peptidases, carboxy peptidase A, etc.), while others attack internal peptide bonds of a protein (endo peptidases, such as trypsin, chymotrypsin, pepsin, papain, elastase, etc.). Proteases are divided into four major groups according to the character of their catalytic active site and conditions of action: serine proteinases, cysteine (thiol) proteinases, aspartic proteinases, and metalloproteinases. Attachment of a protease to a certain group depends on the structure of the catalytic site and the amino acid (as one of the constituents) essential for its activity. GAGases are enzymes capable of hydrolyzing Glycosaminoglycans (GAGs) or mucopolysaccharides which are long unbranched polysaccharides consisting of a repeating disaccharide unit. The repeating unit consists of a hexose (six-carbon sugar) or a hexuronic acid, linked to a hexosamine (six-carbon sugar containing nitrogen). These molecules are an important component of connective tissues. GAG chains may be covalently linked to a protein to form proteoglycans, for example, chondroitins, which can be found in connective tissues, cartilage, and tendons. Metalloproteinases (or metalloproteases) are proteolytic enzymes whose catalytic activity is zinc- or cobalt-dependent. The zinc or cobalt ion found in metalloproteinases is coordinated to the protein via three ligands. The ligands coordinating the metal ion can vary with histidine, glutamate, aspartate, lysine and arginine. The fourth coordination position is taken up by a labile water molecule. There are two subgroups of metalloproteinases and exopeptidases such metalloexopeptidases, endopeptidases and metalloendopeptidases. Metalloendopeptidases include, for example, the matrix metalloproteinases. Clostripain, also known as endoproteinase Arg-C, is a proteinase that cleaves proteins on the carboxyl peptide bond of arginine. It was isolated from Clostridium histolyticum and functions optimally at a pH of about 7.4 to about 7.8.

Enzymatic digestion specifically cleaves bonds joining the PVMCs to the basement membrane of small blood vessels. More specifically, enzymatic digestion cleaves the attachment of a PVMC from a basement membrane of a small blood vessel. In some embodiments, an enzymatic digestion also includes enzymatic digestion to cleave bonds joining the PVMC to molecules that may be themselves bound to the basement membrane. For example, PDGF-BB, which binds to heparin in the basement membrane, and PDGF-BB, in turn, may be capable of binding to PVMCs which express the PDGF receptor. In some embodiments, enzymatic digestion results in the cleavage of specific peptide bonds, ester linkages, or combinations thereof, involving a sugar and a peptide or a sugar and another sugar. In some embodiments, enzymatic digestion can specifically cleave linkages to complex lipids or simple esters of fatty acids. In further embodiments, enzymatic digestion can cleave linkages to cholesterol or molecules where the bond involves a benzene ring. In some embodiments, cleavage of one bond can result in destabilization of other bonds resulting in a conformational change in a molecule that may be associated with a PVMC.

In some embodiments, the enzyme mixture further comprises an antibiotic selected from streptomycin, gentamicin, fungizone, penicillinG, amphotericin B, or a combination thereof. In some embodiments, the antibiotic comprises about 20% of the enzyme mixture. In some embodiments, the enzyme mixture further comprises a medium selected from Tyrode's solution, lactated Ringer's Solution, minimum essential medium Eagle alpha modification (α-MEM), phosphate buffered saline (PBS), or a combination thereof. In some embodiments, the antibiotic in the enzyme mixture may comprise an antibiotic selected from streptomycin, gentamicin, fungizone, penicillinG, amphotericin B or a combination thereof.

In some embodiments, the enzyme mixture is incubated for from about 1 to about 10 minutes, about 10 to about 20 minutes, about 20 to about 60 minutes, about 20 to about 30 minutes, about 30 to about 40 minutes, about 40 to about 50 minutes, about 50 to about 60 minutes, about 60 to about 120 minutes. In some embodiments, the enzyme mixture is incubated at a temperature ranging from about 15° C. to about 38° C., about 15° C. to about 20° C., about 20° C. to about 25° C., about 25° C. to about 30° C., about 30° C. to about 35° C., or about 35° C. to about 38° C. In some embodiments, the disassociating enzyme mixture is inactivated by flushing the umbilical cord blood vessel with phosphate buffered saline (PBS) and the enzyme mixture and PBS are collected.

In some embodiments, a second incubation with a second enzyme mixture may be performed. In some embodiments, multiple incubations with an enzyme mixture may be performed. Surprisingly, when two incubations with enzyme are performed, the second incubation results in the isolation of PVMCs from an umbilical cord blood vessel. While not wishing to be bound by theory, it is believed that a first incubation with the first enzyme mixture will result in disassociation of endothelial cells, leaving the basement membrane exposed. It is believed that in a second incubation with the second enzyme mixture, the PVMCS, which are bound to the basement membrane, may become disassociated from the basement membrane and may be collected.

In some embodiments, the method of isolating PVMCs from an umbilical cord blood vessel further comprises concentrating PVMCs in a cell suspension. In some embodiments, concentrating PVMCs comprises the use of buoyant density sedimentation, filtration, or centrifugation to obtain a population of concentrated, disassociated perivascular medicinal cells.

In some embodiments, preparing the concentrated umbilical cord blood vessel-derived PVMCs can be supplemented by culturing the concentrated PVMCs after concentrating the PVMCs to selectively expand the population of concentrated umbilical cord blood vessel-derived PVMCs. In some embodiments, PVMCs isolated from an umbilical cord blood vessel can be diluted with Dulbecco's Modified Eagle Medium (DMEM) supplemented with about 1 to 20% fetal bovine serum (FBS). In some embodiments, the DMEM mixture may be vigorously vortexed to mechanically disperse the tissue followed by centrifugation in a bench top centrifuge after which the supernatant may be removed. In some embodiments, the remaining cell pellet will be fractionated to collect nucleated cells using Percoll™ followed by a second round of centrifugation without breaking to ensure an intact Percoll™ gradient. The top fraction of the gradient may then be transferred to a new tube and supplemented with DMEM followed by centrifugation. After centrifugation, the supernatant may be removed without disturbing the pellet. In some embodiments, the pellet may then be re-suspended in DMEM and washed several times by centrifugation using DMEM. The resulting PVMC cell suspension may then be ready for expansion or concentration.

In some embodiments, concentrating a population of PVMCs from an umbilical cord blood vessel can be achieved by the use of magnetic beads comprising antibodies with affinity to cell surface antigens on the PVMC. In yet other embodiments, concentration of PVMCs can be performed upon an expanded cell population. It is expected that PVMCs can be passaged only a finite number of times, thereafter experiencing reduced proliferation and differentiation potential. Furthermore, growth characteristics and cell yield of a PVMC preparation are dependent on donor age and vary among individuals. In yet other embodiments, the concentration of a PVMC population can be performed without prior expansion of the cell population.

In some embodiments, the population of concentrated umbilical cord blood vessel-derived PVMCs is not cultured. In some embodiments, PVMCs obtained from an umbilical cord blood vessel are cultured and expanded in medium.

In some embodiments, PVMCs from an umbilical cord blood vessel may be expanded in cell culture. In some embodiments, primary cultures of PVMCs can be seeded at about 10⁷ cells per 100 mm culture dish expanded in DMEM culture medium containing about 10% fetal calf serum, about 2 mM L-glutamine, about 100 units/mL penicillin and about 100 μg/mL streptomycin. PVMCs adhere to the negatively charged culture dish. In some embodiments, the method may further comprise selecting cells adhering to the culture medium. In some embodiments, the method may further comprise rinsing with DMEM and repeating the selection of adherent cells. Following selection of adherent cells, PVMC populations may be further subcultured.

In some embodiments, PVMCs from an umbilical cord blood vessel express cell surface markers CD29, CD105, CD44, CD73, CD146 and CD166 but not hematopoietic and endothelial markers. In some embodiments, the PVMC population is more than 90% pure in terms of antigen expression and viability and express a phenotype of CD73+/CD105+/CD44+/CD29+/SSEA4+/CD45−/CD31−/vWF−/CD14−. In some embodiments, the PVMCs are negative for MHC class I but express MHC class II. In some embodiments, the PVMC population may be less than 90% pure, less than 80% pure, less than 70% pure, less than 60% pure or less than 50% pure. In some embodiments, a purified or impure cell population may be collected in any appropriate medium. In some embodiments, the PVMCs will be about 30% or more of the purified cell population, preferably 50% or more of the purified cell population, more preferably 90% or more of the purified cell population, and most preferably about 95% or more (substantially pure) of the purified cell population.

Cell surface markers can be characterized by flow cytometry after being labeled with various antibodies including those against human CD29, CD105, CD44, CD73, SSEA4, CD45, CD31, vWF, and CD14. Secondary antibodies conjugated with fluorescin are subsequently used.

In some embodiments, PVMCs from an umbilical cord blood vessel can be isolated by FACS sorting. The use of cell surface antigens, such as, without limitation, CD29, CD105, CD44 and CD73, to isolate PVMCs provides a means for the positive immunoselection of PVMC populations, as well as for the phenotypic analysis of PVMC cell populations, for example, flow cytometry. Cells selected for expression of CD29, CD105, CD44 and CD73 antigens may be further purified by selection for other stem cell and progenitor cell markers, including, but not limited to, SSAE4 human embryonic stem stage specific markers.

In some embodiments, the preparation of substantially pure PVMCs from an umbilical cord blood vessel, a subset of umbilical cord blood vessel-derived PVMCs can be separated from other cells on the basis of other surface markers known in the art.

Procedures for separation may include, without limitation, magnetic separation, using antibody-coated magnetic beads, affinity chromatography and “panning” with antibody attached to a solid matrix, e.g. plate, or other convenient technique. Techniques providing accurate separation include, without limitation, fluorescence activated cell sorters, which can have varying degrees of sophistication, such as multiple color channels, low angle and obtuse light scattering detecting channels, impedance channels, etc. Dead cells may be eliminated by selection with dyes associated with dead cells (propidium iodide (PI), LDS). Any technique may be employed which is not unduly detrimental to the viability of the selected cells.

In some embodiments where antibody-coated magnetic beads are used, the antibodies may be conjugated with labels to allow for ease of separation of the particular cell type, e.g. magnetic beads; biotin, which binds with high affinity to avidin or streptavidin; fluorochromes, which can be used with a fluorescence activated cell sorter (FACS); haptens; and the like. Multi-color analyses may be employed with the FACS or in a combination of immunomagnetic separation and flow cytometry. Multi-color analysis is of interest for the separation of cells based on multiple surface antigens, e.g. CD73+, CD105+, CD44+, CD29+ and antibodies recognizing SSAE4 cell markers. Fluorochromes which find use in a multi-color analysis include, without limitation, phycobiliproteins, e.g. phycoerythrin and allophycocyanins; fluorescin; and Texas red. A negative designation may indicate that the level of staining is at or below the brightness of an isotype matched negative control. A dim designation may indicate that the level of staining may be near the level of a negative stain, but may also be brighter than an isotype matched control.

In some embodiments, CD29, CD105, CD44, CD73, SSEA4, CD45, CD31, vWF, and CD14 antibodies are directly or indirectly conjugated to a magnetic reagent, such as a superparamagnetic microparticle (microparticle). Direct conjugation to a magnetic particle may be achieved by use of various chemical linking groups, as known in the art. The antibody may be coupled to the microparticles through side chain amino or sulfhydryl groups and heterofunctional cross-linking reagents. A large number of heterofunctional compounds are available for linking to entities. In some embodiments, the linking group is 3-(2-pyridyldithio) propionic acid N-hydroxysuccinimide ester (SPDP) or 4-(N-maleimidomethyl)-cyclohexane-1-carboxylic acid N-hydroxysuccinimide ester (SMCC) with a reactive sulfhydryl group on the antibody and a reactive amino group on the magnetic particle.

In some embodiments, CD29, CD105, CD44, CD73, SSEA4, CD45, CD31, vWF, and CD14 antibodies are indirectly coupled to the magnetic particles. In some embodiments, the antibody is directly conjugated to a hapten, and hapten-specific, second stage antibodies are conjugated to the particles. In some embodiments, suitable haptens include digoxin, digoxigenin, FITC, dinitrophenyl, nitrophenyl, avidin, biotin, etc. Methods for conjugation of the hapten to a protein are known in the art, and kits for such conjugations are commercially available.

In some embodiments, the amount of antibody necessary to bind a particular cell subset is empirically determined by performing a test separation and analysis. In some embodiments, the cells and antibody are incubated for a period of time sufficient for complexes to form. In some embodiments, the period of time may be at least about 5 min, at least about 10 min, up to about 30 min, or up to about 60 min.

In some embodiments, the cells may additionally be incubated with antibodies or binding molecules specific for cell surface markers known to be present or absent on the PVMCs. For example, in some embodiments, cells expressing CD45, CD31, vWF or CD14 marker can be negatively selected.

In some embodiments, the labeled cells are separated in accordance with the specific antibody preparation. In some embodiments, fluorochrome-labeled antibodies are useful for FACS separation, magnetic particles for immunomagnetic selection, and particularly high gradient magnetic selection (HGMS), etc. Exemplary magnetic separation devices are described in WO 90/07380, PCT/US96/00953, and EP 438,520 each of which is hereby incorporated by reference in their entireties.

In some embodiments, the PVMC cell population from an umbilical cord blood vessel may be collected in any appropriate medium. Various media are commercially available and may be used, including Dulbecco's Modified Eagle Medium (DMEM), Hank's Basic Salt Solution (HBSS), Dulbecco's phosphate buffered saline (dPBS), RPM, Iscove's modified Dulbecco's medium (IMDM), phosphate buffered saline (PBS) with 5 mM EDTA, etc., frequently supplemented with fetal calf serum (FCS), bovine serum albumin (BSA), human serum albumin (HSA), etc. Preferred culture media include DMEM, F-12, Ml99, and RPMI.

In some embodiments, the PVMCs from an umbilical cord blood vessel may comprise about 30% or more of the cell population, about 50% or more of the cell population, about 90% or more of the cell population, or about 95% or more of the cell population.

In some embodiments, isolated PVMCs from an umbilical cord blood vessel may be expanded in DMEM culture medium containing about 10% fetal calf serum, about 2 mM L-glutamine, about 100 units/mL penicillin and about 100 μg/mL streptomycin. In some embodiments, the umbilical cord blood vessel-derived PVMCs are placed into T75 flasks and diluted with DMEM culture medium. In some embodiments, this mixture is stored in an incubator at about 37° C. with about 5% CO₂ for about three days. After the incubation time, the PVMCs adhere to the surface of the flask and the remnant components of bone marrow can be eliminated by washing with PBS.

In some embodiments, PVMCs from an umbilical cord blood vessel can be expanded utilizing basal medium and low glucose along with about 10-20% fetal bovine serum (FBS). In some embodiments, other protein sources such as platelet lysates can be used. In some embodiments, additional factors such as recombinant human fibroblastic growth factor (rhFGF) can be used as a culture supplement to enhance proliferation capacity. In some embodiments, about 10 ng/mL rhFGF-2 is used to reduce population doubling time of PVMC cell populations.

In some embodiments, isolated PVMCs from an umbilical cord blood vessel can be expanded in Iscove's modified Dulbecco's medium (IMDM) supplemented with about 10% fetal bovine serum, about 10 ng/mL fibroblast growth factor, about 2 mM L-glutamine, and about 100 U/L penicillin-streptomycin in a 37° C. incubator with about 5% CO₂ and passaged about 10 to 20 times prior to isolating.

Fetal bovine serum (FBS) may harbor pathogens and PVMC recipients may develop anti-FBS antibodies, requiring in some instances the use of serum-free media. In some embodiments, PVMCs can be grown in serum-free media supplemented with FGF, platelet-derived growth factor (PDGF), and transforming growth factor-β (TGF-β). In some embodiments, platelet-rich plasma may be used as an effective substitute for FBS.

Some embodiments describe a method of isolating PVMCs from an umbilical cord blood vessel comprising draining a content of the umbilical cord blood vessel to make a cell suspension and dissociating the PVMCs from the cell suspension using an enzyme mixture. In some embodiments, the process of draining the contents of the umbilical cord blood vessel comprises inserting a needle into the umbilical cord blood vessel and flushing with a solution. In some embodiments, the method further comprises collecting the cell suspension. In some embodiments, the solution is phosphate buffered saline (PBS), lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), or a combination thereof.

In some embodiments, disassociating the PVMCs is performed upon an intact umbilical cord blood vessel. In some embodiments, a method of isolating PVMCs from an umbilical cord blood vessel comprises washing the umbilical cord blood vessel with a washing fluid and adding an enzyme mixture. In some embodiments, only the interior of the umbilical cord blood vessel is exposed to washing fluid or the enzyme mixture.

In some embodiments, the enzyme mixture is incubated within the umbilical cord blood vessel. In some embodiments, the enzyme mixture comprises collagenases, neutral or acidic proteases, GAGases, metalloproteases clostripain, serine proteases, alkaline proteases, cysteine proteases, or combinations thereof. The enzymes may be, without limitation, from an animal, plant, bacteria, or fungi, or a combination thereof.

Some embodiments are a method of isolating PVMCs from an umbilical cord blood vessel comprising adding an enzyme to the umbilical cord blood vessel to disassociate the PVMCs from the umbilical cord blood vessel. Some embodiments further comprise draining the umbilical cord blood vessel and adding an enzyme to the umbilical cord blood vessel to disassociate the PVMCs from the umbilical cord blood vessel. In some embodiments, the process of draining the contents of the umbilical cord blood vessel comprises flushing the umbilical cord blood vessel with saline. In some embodiments, draining the umbilical cord blood vessel comprises inserting a needle in to the umbilical cord blood vessel and flushing the umbilical cord blood vessel with saline. In some embodiments, the saline is phosphate buffered saline (PBS).

In some embodiments, the enzyme is selected from collagenases, neutral or acidic proteases, GAGases, metalloproteases clostripain, serine proteases, alkaline proteases, cysteine proteases, or combinations thereof. The enzyme may be, without limitation, from an animal, plant, bacteria, or fungi, or a combination thereof.

In some embodiments, the enzyme mixture further comprises an antibiotic selected from streptomycin, gentamicin, fungizone, penicillinG, amphotericin B, or a combination thereof. In some embodiments, the antibiotic comprises about 20% of the enzyme mixture. In some embodiments, the enzyme mixture further comprises a medium selected from Tyrode's solution, lactated Ringer's Solution, minimum essential medium Eagle alpha modification (α-MEM), phosphate buffered saline (PBS), or a combination thereof. In some embodiments, the antibiotic in the enzyme mixture may comprise an antibiotic selected from streptomycin, gentamicin, fungizone, penicillinG, amphotericin B or a combination thereof.

Some embodiments comprise incubating the enzyme with the umbilical cord blood vessel. In some embodiments, the enzyme is incubated for about 20 to about 60 minutes. In some embodiments, the enzyme is incubated at a temperature ranging from about 15° C. to about 35° C.

Some embodiments further comprise inactivating the enzyme. In some embodiments, inactivating the enzyme comprises flushing the umbilical cord blood vessel with a solution. In some embodiments, the solution may be selected from phosphate buffered saline (PBS), serum containing medium, EDTA, Diisopropylfluorophosphate (DFP), Ethylenediaminetetraacetic acid (EDTA), ethylene glycol tetraacetic acid (EGTA), cysteine, histidine, Dithiothreitol (DTT), 2-mercaptoethanol, o-phenanthroline, Hg2+, Pb2+, Cd2+, Cu2+, Zn2+, Co2+, α2-macroglobulin, 1,10-phenanthroline, Tosyl Lysyl Chloromethyl Ketone (TLCK), heavy metal ions, Citrate, borate and Tris anions, alpha 1-antitrypsin, C1-inhibitor, antithrombin, alpha 1-antichymotrypsin, plasminogen activator inhibitor-1, neuroserpin, aprotinin, bestatin, E64, Leupeptin, tissue inhibitors of metalloproteinases (TIMPs) 1-4, or a combination thereof.

Some embodiments further comprise incubating the umbilical cord blood vessel with an enzyme mixture for the second time. In some embodiments, the enzyme mixture may be the same enzyme as used in the first incubation.

In some embodiments, isolating PVMCs from an umbilical cord blood vessel is performed in a sterile environment.

Some embodiments further comprise concentrating a population of perivascular medicinal cells. In some embodiments, concentrating a population of PVMCs comprises buoyant density sedimentation, filtration, centrifugation, or a combination thereof. In some embodiments, concentrating the population of PVMCs yields a population of concentrated, disaggregated perivascular medicinal cells.

Some embodiments are a method of isolating PVMCs whereby multiple umbilical cord blood vessel derived preparations are combined. In some embodiments, multiple umbilical cord blood vessel derived preparations can be combined following the step of concentrating a population of perivascular medicinal cells. In some embodiments, multiple umbilical cord blood vessel derived preparations can be combined following the step of performing a second incubation with a second enzyme.

Some embodiments are a method of isolating PVMCs from an umbilical cord blood vessel comprising adding an enzyme mixture to the blood vessel to disassociate the perivascular medicinal cells. In some embodiments, the umbilical cord blood vessel may be from about 2 cm to about 10 cm in length. In some embodiments, the blood vessel is a vein or artery. In some embodiments, the enzyme mixture comprises a medium selected from Tyrode's solution, lactated Ringer's Solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof. In some embodiments, the antibiotic in the enzyme mixture may comprise an antibiotic selected from streptomycin, gentamicin, fungizone, penicillinG, amphotericin B or a combination thereof. In some embodiments, the antibiotic comprises about 20% of the enzyme mixture. In some embodiments, the enzyme mixture comprises collagenases, neutral or acidic proteases, GAGases, metalloproteases clostripain, serine proteases, alkaline proteases, cysteine proteases, or combinations thereof. The enzymes may be, without limitation, from an animal, plant, bacteria, or fungi, or a combination thereof. In some embodiments, the enzyme mixture comprises type IV collagenase.

In some embodiments, the umbilical cord blood vessel is immersed in an immersion medium prior to addition of the enzyme mixture comprising medium, antibiotics, or a combination thereof. In some embodiments, the immersion medium comprises Tyrode's solution, lactated Ringer's Solution, acetated Ringer's solution, TRIS-buffered saline (TBS), Hank's balanced salt solution (HBSS), Earle's balanced salt solution (EBSS), Standard saline citrate (SSC), HEPES-buffered saline (FIBS), Gey's balanced salt solution (GBSS), minimum essential medium Eagle alpha modification (α-MEM), phosphate buffered saline (PBS), or a combination thereof. In some embodiments, the antibiotic may be selected from streptomycin, gentamicin, fungizone, penicillinG, amphotericin B, or a combination thereof. In some embodiments, the antibiotic may comprise about 20% of the immersion medium.

In some embodiments, the umbilical cord blood vessel may be canulated. Some embodiments further comprise washing the canulated blood vessel with a heparin medium. In some embodiments, the heparin medium may be Tyrode's solution, lactated Ringer's Solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof. In some embodiments, the heparin medium comprises heparin in an amount from about 50 to about 200 units/mL.

Some embodiments further comprise clamping a first end of the umbilical cord blood vessel before adding the blood vessel with the enzyme mixture. Some embodiments further comprise clamping a second end of the umbilical cord blood vessel after the enzyme mixture is added to the blood vessel to make a clamped umbilical cord blood vessel. Some embodiments further comprise incubating the clamped umbilical cord blood vessel at a temperature ranging from about 15° C. to about 38° C. after making a clamped umbilical cord blood vessel. In some embodiments, the clamped umbilical cord blood vessel may be incubated for about 15 to about 60 minutes.

Some embodiments further comprise unclamping a first end of the umbilical cord blood vessel and adding Tyrode's solution to the umbilical cord blood vessel. Some embodiments further comprise reclamping the first end of the umbilical cord blood vessel after adding Tyrode's solution and massaging the umbilical cord blood vessel. Some embodiments further comprise collecting a wash eluent after adding Tyrode's solution. In some embodiments, the wash eluent comprises a cell suspension of endothelial and subendothelial cells. Some embodiments further comprise washing the eluent with medium. In some embodiments, the medium may be selected from Tyrode's solution, lactated Ringer's Solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof

Some embodiments further comprise adding a second enzyme mixture to the umbilical cord blood vessel. In some embodiments, the enzyme mixture comprises an enzyme, a medium, an antibiotic, or a combination thereof. In some embodiments, the medium may be selected from Tyrode's solution, lactated Ringer's Solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), minimum essential medium Eagle alpha modification (α-MEM), phosphate buffered saline (PBS), or a combination thereof.

In some embodiments, the antibiotic may be selected from streptomycin, gentamicin, fungizone, penicillinG, amphotericin B, or a combination thereof. In some embodiments, the antibiotic comprises about 20% of the enzyme mixture. In some embodiments, the enzyme may be selected from collagenases, neutral or acidic proteases, GAGases, metalloproteases clostripain, serine proteases, alkaline proteases, cysteine proteases, or combinations thereof. The enzymes may be, without limitation, from an animal, plant, bacteria, or fungi, or a combination thereof. In some embodiments, the enzyme comprises type IV collagenase.

Some embodiments further comprise clamping a first end of the umbilical cord blood vessel before adding the enzyme mixture. Some embodiments further comprise clamping a second end of the umbilical cord blood vessel after the enzyme mixture is added to the blood vessel to make a clamped umbilical cord blood vessel. Some embodiments further comprise incubating the clamped umbilical cord blood vessel at a temperature ranging from about 15° C. to about 38° C. after making a clamped umbilical cord blood vessel. In some embodiments, the clamped umbilical cord blood vessel may be incubated for about 15 to about 60 minutes.

Some embodiments further comprise unclamping a first end of the umbilical cord blood vessel and adding Tyrode's solution to the umbilical cord blood vessel. Some embodiments further comprise reclamping the first end of the umbilical cord blood vessel after adding Tyrode's solution and massaging the umbilical cord blood vessel. Some embodiments further comprise collecting a wash eluent after adding Tyrode's solution.

Some embodiments are directed towards a composition comprising PVMCs isolated from an umbilical cord blood vessel. In some embodiments, the PVMCs have medicinal capabilities.

Methods for Isolating PVMCs from Bone

In some embodiments, a method of isolating PVMCs from bone comprises extracting a cell suspension from the bone and separating a population of PVMCs from the cell suspension. In some embodiments, extracting comprises enzymatic digestion, mechanical force, or a combination thereof. In some embodiments, the method further comprises concentrating the population of PVMCs. In some embodiments, the method further comprises selectively expanding a population of the concentrated PVMCs.

In some embodiments, PVMCs may be isolated from bone chips. In some embodiments, PVMCs may be isolated from bone marrow scoops, bone marrow scopes or bone cavity lavages.

In some embodiments, isolating PVMCs from bone comprises (i) extracting a cell suspension from the bone by enzymatic digestion, mechanical force, or a combination thereof; and (ii) separating a population of PVMCs from the cell suspension by buoyant density sedimentation, filtration, centrifugation, or a combination thereof.

In some embodiments, extracting a cell suspension from the bone comprises enzymatic digestion to specifically cleave bonds joining the PVMCs to a basement membrane of small blood vessels in the bone. More specifically, enzymatic digestion may cleave molecules that may be part of the basement membrane, releasing PVMC binding domains as a result.

In some embodiments, the method further comprises grinding the bone before extracting the cell suspension. In some embodiments, grinding comprises cleaning the bone to remove extraneous soft tissue and grinding the bone. In some embodiments, particle sizes range from about 1 to about 50 mm³. In some embodiments, enzymatic digestion of the bone may comprise treating the bone with enzymes selected from collagenases neutral or acidic proteases, GAGases, metalloproteases, clostripain (a cysteine protease from C. histolyticum), serine proteases, alkaline proteases, cysteine proteases, or combinations thereof. The enzymes may be, without limitation, from an animal, plant, bacteria, or fungi, or a combination thereof. In some embodiments, the enzymatic digestion specifically cleaves bonds joining the PVMCs to the basement membrane of small blood vessels within the bone fragments. In some embodiments, enzymatic digestion cleaves bonds of the basement membrane that house separated, associated molecules to which the PVMCs separately associate; for example, PDGF-BB which binds to heparin in the basement membrane, and PDGF-BB, in turn, may be capable of binding to PVMCs which express the PDGF receptor. In some embodiments, the enzymatic digestion of bone fragments can be performed subsequently to mechanical breakdown of bone as well as with intact fragments of bone.

In some embodiments, extracting the cell suspension from bone may comprise density gradient centrifugation. In some embodiments, density gradient centrifugation may be accomplished by serial centrifugation steps. In some embodiments, centrifugation may be performed at about 500×g to about 2,500×g. In some embodiments, these preparations may be suitable for direct use in orthopedic and dental applications. In some embodiments, the bone may be subjected to enzymatic digestion prior to density gradient centrifugation.

In some embodiments, extracting the PVMCs from bone may be achieved by diluting a bone marrow sample with phosphate buffered saline (PBS). Dilution in a suitable medium such as PBS provides a stable environment for the process of enzymatic digestion. In some embodiments, dilution in PBS may be followed by the step of enzymatic digestion using one or more enzymes such as collagenases, neutral or acidic proteases, GAGases, or metalloproteases, clostripain (a cysteine protease from C. histolyticum), serine proteases, alkaline proteases, cysteine proteases, or combinations thereof. The one or more enzymes may be, without limitation, from an animal, plant, bacteria, or fungi, or a combination thereof. In some embodiments, the enzymatic digestion may use an enzyme that cleaves the attachment of a PVMC from a basement membrane of a small blood vessel or sinusoid. In some embodiments, the extraction of PVMCs from bone marrow does not include a step of enzymatic digestion. In some embodiments, the diluted or enzymatically digested sample may be then subjected to density gradient separation using Percoll™ to obtain mononuclear cells. The mononuclear fraction may be plated in cell culture cassettes in Dulbecco's Modified Eagle Medium (DMEM) for expansion of PVMCs. In some embodiments, the method further comprises separating a population of PVMCs from the cell suspension by buoyant density sedimentation, filtration, centrifugation, immuno-bead selection, or a combination thereof.

In some embodiments, the method may further comprise culturing the concentrated PVMCs to selectively expand a population of concentrated bone-derived PVMCs.

In some embodiments, a bone chip or milled bone sample comprising bone tissue may be diluted with DMEM supplemented with 1 to 20% fetal bovine serum (FBS). In some embodiments, the bone-DMEM mixture may be vigorously vortexed to mechanically disperse the tissue and separate it from the bone followed by centrifugation in a bench top centrifuge after which the supernatant may be removed. In some embodiments, the remaining cell pellet will be fractionated to collect nucleated cells using Percoll™ followed by a second round of centrifugation without breaking to ensure an intact Percoll™ gradient. The top fraction of the gradient may then be transferred to a new tube and supplemented with DMEM followed by centrifugation. After centrifugation, the supernatant may be removed without disturbing the pellet. In some embodiments, the pellet may be then resuspended in DMEM and washed several times by centrifugation using DMEM. The resulting PVMC cell suspension may then be ready for expansion, concentration or may be used therapeutically. In some embodiments, the PVMC cell suspension may be combined with bone. In some embodiments, the PVMC cell suspension may be delivered systemically or injected directly into a subject in need thereof.

In some embodiments, concentrating the population of PVMCs comprises using magnetic beads comprising antibodies with affinity to cell surface antigens on the PVMC. In some embodiments, concentrating PVMCs may comprise concentrating an expanded cell population. Without wishing to be bound by theory, it is believed that PVMCs may be passaged only a (mite number of times, thereafter experiencing reduced proliferation and differentiation potential. Furthermore, it is believed that growth characteristics and cell yield of a PVMC preparation may be dependent on donor age and vary among individuals. In some embodiments, the concentration of a PVMC population may be performed without prior expansion of the cell population.

In some embodiments, the population of concentrated bone-derived PVMCs may not be cultured. In some embodiments, PVMCs obtained from human bone marrow may be cultured and expanded in medium.

In some embodiments, bone-derived PVMCs may be expanded in cell culture. Primary cultures of PVMCs can be seeded at about 10⁵ to about 10⁹ cells per 100 mm culture dish and expanded in DMEM culture medium containing about 1 to 20% fetal calf serum, about 1-3 mM L-glutamine, about 5-200 units/mL penicillin and about 5-200 μg/mL streptomycin. PVMCs may adhere to the negatively charged culture dish allowing for selection of only adherent cells following repeated passages and rinses with DMEM. In some embodiments, PVMC populations can be further subcultured following selection of adherent cells. In some embodiments, PVMCs may be selected by pre-coating culture dishes with human fibronectin. In some embodiments, human fibronectin may be added to the culture medium. In some embodiments, adherent PVMCs may be removed from a culture dish by treatment with trypsin. In some embodiments, such selective culturing removes cells of hematopoietic function because these cells are non-adherent or attach poorly. In some embodiments, hematopoietic cells adhere to the culture medium and do not detach upon treatment with trypsin.

Bone-derived PVMCs may express a cell surface marker selected from CD29, CD105, CD44, CD73, CD146, CD166, or any combination thereof. In some embodiments, the PVMCs may not express hematopoietic and endothelial markers. In some embodiments, isolated PVMC populations of embodiments herein may be greater than about 90% pure. In some embodiments, PVMC populations of embodiments herein may be greater than about 50% pure, 60% pure, 70% pure, or 80% pure. In some embodiments, the term “pure” refers to antigen expression, viability, ability to express a phenotype of CD73+/CD105+/CD44+/CD29+/SSEA4+/CD45−/CD31−/vWF−/CD14−, or any combination thereof. In some embodiments, isolated PVMC populations of embodiments herein may be up to about 90% pure, up to about 80% pure, up to about 70% pure, up to about 60% pure or up to about 50% pure. In some embodiments, bone-derived PVMC populations are impure.

Cell surface markers may be characterized by flow cytometry after being labeled with various antibodies including those against human CD29, CD105, CD44, CD73, SSEA4, CD45, CD31, vWF, and CD14. Secondary antibodies conjugated with fluorescin may be subsequently used.

In some embodiments, bone-derived PVMCs may be extracted and concentrated from a heterogeneous bone-derived cell preparation or by FACS sorting. The use of cell surface antigens to PVMCs, such as CD29, CD105, CD44, CD73, provides a means for the positive immunoselection of PVMC populations, as well as for the phenotypic analysis of PVMC cell populations, for example, flow cytometry. Cells selected for expression of CD29, CD105, CD44 and CD73 antigens may be further purified by selection for other stem cell and progenitor cell markers, including, but not limited to, SSAE4 human embryonic stem stage specific markers.

In some embodiments, concentrating a population of bone-derived PVMCs from the cell suspension comprises separating the PVMCs from the heterogeneous cell suspension using surface markers of the PVMCs. In some embodiments, the surface markers used for separating the PVMCs may include CD29, CD105, CD44, CD73, SSAE4, or any combination thereof.

In some embodiments, procedures for extracting and concentrating bone-derived PVMCs may include magnetic separation, using antibody-coated magnetic beads, affinity chromatography and “panning” with antibody attached to a solid matrix, e.g. plate, or other convenient technique known in the art. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells, e.g. propidium iodide (PI), or LDS. Any technique may be employed which is not unduly detrimental to the viability of the selected cells.

In some embodiments, antibody-coated magnetic beads may comprise antibodies conjugated with labels to allow for ease of separation of the particular cell type, e.g. magnetic beads; biotin, which binds with high affinity to avidin or streptavidin; fluorochromes, which can be used with a fluorescence activated cell sorter (FACS); haptens; or the like. Multi-color analysis may be employed with the FACS or in a combination of immunomagnetic separation and flow cytometry. Multi-color analysis may be of interest for the separation of cells based on multiple surface antigens, e.g. CD73+, CD105+, CD44+, CD29+ and antibodies recognizing SSAE4 cell markers. Fluorochromes which find use in a multi-color analysis may include, without limitation, phycobiliproteins, e.g. phycoerythrin and allophycocyanins; fluorescein and Texas red. In some embodiments, a negative designation indicates that the level of staining may be at or below the brightness of an isotype matched negative control. In some embodiments, a dim designation indicates that the level of staining may be near the level of a negative stain, but may also be brighter than an isotype matched control.

In some embodiments, CD29, CD105, CD44, CD73, SSEA4, CD45, CD31, vWF, and CD14 antibodies may be directly or indirectly conjugated to a magnetic reagent, such as a superparamagnetic microparticle. Direct conjugation to a magnetic particle may be achieved by use of various chemical linking groups. In some embodiments, antibodies can be coupled to the microparticles through side chain amino or sulfhydryl groups and heterofunctional cross-linking reagents. A large number of heterofunctional compounds may be available for linking to entities. In some embodiments, the linking group may include, without limitation, 3-(2-pyridyldithio) propionic acid N-hydroxysuccinimide ester (SPDP) or 4-(N-maleimidomethyl)-cyclohexane-1-carboxylic acid N-hydroxysuccinimide ester (SMCC) with a reactive sulfhydryl group on the antibody and a reactive amino group on the magnetic particle.

In some embodiments, anti-CD29, anti-CD105, anti-CD44, anti-CD73, anti-SSEA 4, anti-CD45, anti-CD31, anti-vWF, and anti-CD14 antibodies may be indirectly coupled to the magnetic particles. The antibody may be directly conjugated to a hapten, and hapten-specific, second stage antibodies may be conjugated to the particles. Suitable haptens include, without limitation, digoxin, digoxigenin, FITC, dinitrophenyl, nitrophenyl, avidin, biotin, etc. Methods for conjugation of the hapten to a protein are known in the art and kits for such conjugations are commercially available.

In some embodiments, the amount of antibodies necessary to bind a particular cell subset may be empirically determined by performing a test separation and analysis. In some embodiments, the cells and antibodies may be incubated for a period of time sufficient for complexes to form, for example, without limitation, at least about 5 min, at least about 10 min, or about 30 min or less, or about 60 minutes or less, or a range between any two of these values.

In some embodiments, the cells may additionally be incubated with antibodies or binding molecules specific for cell surface markers known to be present or absent on the PVMCs. For example, cells expressing CD45, CD31, vWF, or CD14 markers can be negatively selected for.

In some embodiments, the labeled cells may be separated in accordance with the specific antibody preparation. Fluorochrome labeled antibodies may be useful for FACS separation, magnetic particles for immunomagnetic selection, particularly high gradient magnetic selection (HGMS), etc. In embodiments where procedures for separating and isolating PVMCs use antibodies, it should be noted that, in some embodiments, such antibodies may be consumed by natural cellular processes during cell culture and expansion such that no antibodies will be detectable in the PVMC preparations that may be used in enhanced autologous bone grafts.

In some embodiments, a purified or impure cell population may be collected in any appropriate medium. In some embodiments, any commercially available media may be used, including, without limitation, Dulbecco's Modified Eagle Medium (DMEM), Hank's Basic Salt Solution (HBSS), Dulbecco's phosphate buffered saline (dPBS), RPMI, Iscove's modified Dulbecco's medium (IMDM), phosphate buffered saline (PBS) with about 5 mM EDTA, etc., frequently supplemented with fetal calf serum (FCS), bovine serum albumin (BSA), human serum albumin (HSA), or a combination thereof. In some embodiments, the medium may be DMEM, F-12, Ml99, RPMI, or any combination thereof.

In some embodiments, the PVMCs will be about 30% or more of the purified cell population, preferably 50% or more of the purified cell population, more preferably 90% or more of the purified cell population, and most preferably about 95% or more (substantially pure) of the purified cell population.

In some embodiments, isolated PVMCs can be expanded in DMEM culture medium containing about 10% fetal calf serum, about 2 mM L-glutamine, about 100 units/mL penicillin and about 100 μg/mL streptomycin. The bone marrow may be placed into T75 flasks and diluted with DMEM culture medium. This mixture may be stored in an incubator at about 37° C. with about 5% CO₂ for about 3 days. After the incubation time, the PVMCs may be adhered to the surface of the flask and the remnant components of bone marrow can be eliminated by washing with PBS.

In some embodiments, PVMCs may be expanded utilizing basal medium and low glucose along with about 1-20% fetal bovine serum (FBS). In some embodiments, other protein sources such as platelet lysates can be used. In some embodiments, additional factors such as, without limitation, recombinant human fibroblastic growth factor (rhFGF) as a culture supplement to enhance proliferation capacity can be used. In some embodiments, 10 ng/mL rhFGF-2 may reduce population doubling time of PVMC cell populations.

In another embodiment, isolated PVMCs can be expanded in Iscove's modified Dulbecco's medium (IMDM) supplemented with about 1-200% fetal bovine serum, about 1-20 ng/mL fibroblast growth factor, about 1-3 mM L-glutamine and about 1-200 U/L penicillin-streptomycin in a 37° C. incubator with about 5% CO₂ and passaged about 5-30 times prior to isolating.

Fetal bovine serum may harbor pathogens and PVMC recipients may develop anti-FBS antibodies, requiring in some instances the use of serum-free media. In some embodiments, PVMCs can be grown in serum-free media supplemented with FGF, platelet-derived growth factor (PDGF) and transforming growth factor-β (TGF-β). In further embodiments, platelet-rich plasma also appears an effective substitute for FBS. Autologous serum also represents a viable alternative, though limited by the large volume necessary to supplement the media. In addition, several serum-free defined media are commercially available for the expansion for mesenchymal stem cells that will also be applicable to the expansion of PVMCs. In some embodiments, autologous serum can be used to supplement serum free media. In some embodiments, autologous serum is added to serum-free media to achieve a final concentration of about 5 to about 25%. In some embodiments, the autologous serum may be autologous human serum. The use of such chemically defined media requires that they be optimized for a particular source of PVMC and, in some cases, for a particular therapeutic use for PVMCs.

Some embodiments are directed to a method of isolating PVMCs obtained from bone, the method comprising: (i) providing a sample of bone or bone tissue from a subject; (ii) extracting the PVMCs from the bone or bone tissue; and (iii) concentrating the extracted PVMCs.

In some embodiments, bone or bone tissue may be processed by passage through a grinder to produce bone chips. Bone chips may comprise both bone and marrow tissue. In some embodiments, a bone chip may comprise compact bone, bone marrow, tissue from the medullary canal, cancellous tissue, or combinations thereof. In some embodiments, fragments of bone are milled to bone chips by passage through a bone mill such as a Noviomagus Bone Mill. In some embodiments, bone fragments are frozen prior to milling. In some embodiments, bone fragments are fresh prior to milling. In some embodiments, the milled bone chips have an intact trabecular structure. In some embodiments, PVMCs can be cultured to selectively expand a PVMC population. In some embodiments, PVMCs may not be cultured. In some embodiments, the cultured PVMCs may be adherent to cell culture surfaces.

In some embodiments, extracting the PVMCs comprises: (i) extracting a cell suspension from the bone, by enzymatic digestion, mechanical force, or a combination thereof; and (ii) separating a population of PVMCs from the cell suspension by buoyant density sedimentation, filtration, centrifugation, or a combination thereof.

In some embodiments, formation of a cell suspension from the bone comprises enzymatic digestion to specifically cleave bonds joining the PVMCs to a basement membrane of small blood vessels in the bone. In some embodiments, enzymatic digestion comprises cleavage of PVMCs from a basement membrane surrounding small blood vessels. In some embodiments, enzymatic digestion cleaves bonds of the basement membrane that house separated, associated molecules to which the PVMCs separately associate, for example, PDGF-BB which binds to heparin in the basement membrane, and PDGF-BB in turn may be capable of binding to PVMCs which express the PDGF receptor.

In some embodiments, the enzymatic digestion may be achieved by using one or more enzymes selected from collagenase, neutral or acidic proteases, GAGases, or metalloproteases, clostripain, serine proteases, alkaline proteases, cysteine proteases, or combinations thereof. The one or more enzymes may be, without limitation, from an animal, plant, bacteria, or fungi, or a combination thereof. In some embodiments, the enzymatic digestion uses one or more enzymes that cleave the attachment of a PVMC from a basement membrane of a small blood vessel. In some embodiments, enzymatic digestions also include enzymatic digestion to cleave bonds joining the PVMC to molecules that may be themselves bound to the basement membrane. For example, PDGF-BB which binds to heparin in the basement membrane and PDGF-BB, in turn, may be capable of binding to PVMCs which express the PDGF receptor.

In some embodiments, the method of isolating PVMCs further comprises a step of concentrating a population of PVMCs. In some embodiments, concentrating a population of PVMCs may be achieved by the use of magnetic beads comprising antibodies with affinity to cell surface antigens on the PVMC. In some embodiments, antibodies are selected from anti-CD146, anti-CD105, anti-CD166, anti-CD271, or a combination thereof. In some embodiments, antibodies with an affinity or CD45, CD34 or a combination thereof can be used to remove cells expressing CD45, CD34, or a combination thereof from the population of PVMCs. In some embodiments, an antibody affinity column can be used through which PVMC preparations may be passed and then subsequently eluted to generate a more concentrated PVMC preparation.

In some embodiments, preparing the concentrated bone-derived PVMCs further comprises culturing the concentrated PVMCs after concentrating the PVMCs to selectively expand the population of concentrated bone-derived PVMCs. In some embodiments, the population of concentrated bone-derived PVMCs is not cultured.

In some embodiments, concentrating the cells in the cell suspension may be by centrifugation.

### Methods for Concentrating and Expanding Isolated PVMCs

In some embodiments, concentrating the population of isolated PVMCs comprises using magnetic beads comprising antibodies with affinity to cell surface antigens on the isolated PVMC. In some embodiments, concentrating the isolated PVMCs may comprise concentrating an expanded cell population. Without wishing to be bound by theory, it is believed that isolated PVMCs may be passaged only a finite number of times, thereafter experiencing reduced proliferation and differentiation potential. Furthermore, it is believed that growth characteristics and cell yield of an isolated PVMC preparation may be dependent on donor age and vary among individuals. In some embodiments, the concentration of an isolated PVMC population may be performed without prior expansion of the cell population.

In some embodiments, isolated PVMCs may be expanded in cell culture. Primary cultures of isolated PVMCs can be seeded at about 10⁵ to about 10⁹ cells per 100 mm culture dish and expanded in DMEM culture medium containing about 1 to 20% fetal calf serum, about 1-3 mM L-glutamine, about 5-200 units/mL penicillin and about 5-200 μg/mL streptomycin. In some embodiments, isolated PVMCs may adhere to the negatively charged culture dish allowing for selection of only adherent cells following repeated passages and rinses with DMEM. In some embodiments, isolated PVMC populations can be further subcultured following selection of adherent cells. In some embodiments, isolated PVMCs may be selected by pre-coating culture dishes with human fibronectin. In some embodiments, human fibronectin may be added to the culture medium. In some embodiments, adherent isolated PVMCs may be removed from a culture dish by treatment with trypsin. In some embodiments, such selective culturing removes cells of hematopoietic function because these cells are non-adherent or attach poorly. In some embodiments, hematopoietic cells adhere to the culture medium and do not detach upon treatment with trypsin.

In some embodiments, isolated PVMCs may express a cell surface marker selected from CD29, CD105, CD44, CD73, CD146, CD166, or any combination thereof. In some embodiments, the PVMCs may not express hematopoietic and endothelial markers. In some embodiments, isolated PVMC populations of embodiments herein may be greater than about 90% pure. In some embodiments, PVMC populations of embodiments herein may be greater than about 50% pure, 60% pure, 70% pure, or 80% pure. In some embodiments, the term “pure” refers to antigen expression, viability, ability to express a phenotype of CD73+/CD105+/CD44+/CD29+/SSEA4+/CD45−/CD31−/vWF−/CD14−, or any combination thereof. In some embodiments, isolated PVMC populations of embodiments herein may be up to about 90% pure, up to about 80% pure, up to about 70% pure, up to about 60% pure or up to about 50% pure. In some embodiments, bone-derived PVMC populations are impure.

Cell surface markers may be characterized by flow cytometry after being labeled with various antibodies including those against human CD29, CD105, CD44, CD73, SSEA4, CD45, CD31, vWF, and CD14. Secondary antibodies conjugated with fluorescin may be subsequently used.

In some embodiments, isolated PVMCs may be extracted and concentrated from a heterogeneous bone-derived cell preparation or by FACS sorting. The use of cell surface antigens to PVMCs, such as CD29, CD105, CD44, CD73, provides a means for the positive immunoselection of PVMC populations, as well as for the phenotypic analysis of PVMC cell populations, for example, flow cytometry. Cells selected for expression of CD29, CD105, CD44 and CD73 antigens may be further purified by selection for other stem cell and progenitor cell markers, including, but not limited to, SSAE4 human embryonic stem stage specific markers.

In some embodiments, concentrating a population of isolated PVMCs from the cell suspension comprises separating the PVMCs from the heterogeneous cell suspension using surface markers of the PVMCs. In some embodiments, the surface markers used for separating the PVMCs may include CD29, CD105, CD44, CD73, SSAE4, or any combination thereof.

In some embodiments, procedures for extracting and concentrating isolated PVMCs may include magnetic separation, using antibody-coated magnetic beads, affinity chromatography and “panning” with antibody attached to a solid matrix, e.g. plate, or other convenient technique known in the art. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells, e.g. propidium iodide (PI), or LDS. Any technique may be employed which is not unduly detrimental to the viability of the selected cells.

In some embodiments, antibody-coated magnetic beads may comprise antibodies conjugated with labels to allow for ease of separation of the particular cell type, e.g. magnetic beads; biotin, which binds with high affinity to avidin or streptavidin; fluorochromes, which can be used with a fluorescence activated cell sorter (FACS); haptens; or the like. Multi-color analysis may be employed with the FACS or in a combination of immunomagnetic separation and flow cytometry. Multi-color analysis may be of interest for the separation of cells based on multiple surface antigens, e.g. CD73+, CD105+, CD44+, CD29+ and antibodies recognizing SSAE4 cell markers. Fluorochromes which find use in a multi-color analysis may include, without limitation, phycobiliproteins, e.g. phycoerythrin and allophycocyanins; fluorescein and Texas red. In some embodiments, a negative designation indicates that the level of staining may be at or below the brightness of an isotype matched negative control. In some embodiments, a dim designation indicates that the level of staining may be near the level of a negative stain, but may also be brighter than an isotype matched control.

In some embodiments, CD29, CD105, CD44, CD73, SSEA4, CD45, CD31, vWF, and CD14 antibodies may be directly or indirectly conjugated to a magnetic reagent, such as a superparamagnetic microparticle. Direct conjugation to a magnetic particle may be achieved by use of various chemical linking groups. In some embodiments, antibodies can be coupled to the microparticles through side chain amino or sulfhydryl groups and heterofunctional cross-linking reagents. A large number of heterofunctional compounds may be available for linking to entities. In some embodiments, the linking group may include, without limitation, 3-(2-pyridyldithio) propionic acid N-hydroxysuccinimide ester (SPDP) or 4-(N-maleimidomethyl)-cyclohexane-1-carboxylic acid N-hydroxysuccinimide ester (SMCC) with a reactive sulfhydryl group on the antibody and a reactive amino group on the magnetic particle.

In some embodiments, anti-CD29, anti-CD105, anti-CD44, anti-CD73, anti-SSEA 4, anti-CD45, anti-CD31, anti-vWF, and anti-CD14 antibodies may be indirectly coupled to the magnetic particles. The antibody may be directly conjugated to a hapten, and hapten-specific, second stage antibodies may be conjugated to the particles. Suitable haptens include, without limitation, digoxin, digoxigenin, FITC, dinitrophenyl, nitrophenyl, avidin, biotin, etc. Methods for conjugation of the hapten to a protein are known in the art and kits for such conjugations are commercially available.

In some embodiments, the amount of antibodies necessary to bind a particular cell subset may be empirically determined by performing a test separation and analysis. In some embodiments, the cells and antibodies may be incubated for a period of time sufficient for complexes to form, for example, without limitation, at least about 5 min, at least about 10 min, or about, about 30 min or less, or about 60 minutes or less, or a range between any two of these values.

In some embodiments, the cells may additionally be incubated with antibodies or binding molecules specific for cell surface markers known to be present or absent on the PVMCs. For example, cells expressing CD45, CD31, vWF, or CD14 markers can be negatively selected for.

In some embodiments, the labeled cells may be separated in accordance with the specific antibody preparation. Fluorochrome labeled antibodies may be useful for FACS separation, magnetic particles for immunomagnetic selection, particularly high gradient magnetic selection (HGMS), etc. In embodiments where procedures for separating and isolating PVMCs use antibodies, it should be noted that, in some embodiments, such antibodies may be consumed by natural cellular processes during cell culture and expansion such that no antibodies will be detectable in the PVMC preparations that may be used in enhanced autologous bone grafts.

In some embodiments, a purified or impure cell population may be collected in any appropriate medium. In some embodiments, any commercially available media may be used, including, without limitation, Dulbecco's Modified Eagle Medium (DMEM), Hank's Basic Salt Solution (HBSS), Dulbecco's phosphate buffered saline (dPBS), RPMI, Iscove's modified Dulbecco's medium (IMDM), phosphate buffered saline (PBS) with about 5 mM EDTA, etc., frequently supplemented with fetal calf serum (FCS), bovine serum albumin (BSA), human serum albumin (HSA), or a combination thereof. In some embodiments, the medium may be DMEM, F-12, Ml99, RPMI, or any combination thereof.

In some embodiments, the isolated PVMCs will be about 30% or more of the purified cell population, preferably 50% or more of the purified cell population, more preferably 90% or more of the purified cell population, and most preferably about 95% or more (substantially pure) of the purified cell population.

In some embodiments, isolated PVMCs can be expanded in DMEM culture medium containing about 10% fetal calf serum, about 2 mM L-glutamine, about 100 units/mL penicillin and about 100 μg/mL streptomycin. The bone marrow may be placed into T75 flasks and diluted with DMEM culture medium. This mixture may be stored in an incubator at about 37° C. with about 5% CO₂ for about 3 days. After the incubation time, the PVMCs may be adhered to the surface of the flask and the remnant components of bone marrow can be eliminated by washing with PBS.

In some embodiments, isolated PVMCs may be expanded utilizing basal medium and low glucose along with about 1-20% fetal bovine serum (FBS). In some embodiments, other protein sources such as platelet lysates can be used. In some embodiments, additional factors such as, without limitation, recombinant human fibroblastic growth factor (rhFGF) as a culture supplement to enhance proliferation capacity can be used. In some embodiments, 10 ng/mL rhFGF-2 may reduce population doubling time of PVMC cell populations.

In another embodiment, isolated PVMCs can be expanded in Iscove's modified Dulbecco's medium (IMDM) supplemented with about 1-200% fetal bovine serum, about 1-20 ng/mL fibroblast growth factor, about 1-3 mM L-glutamine and about 1-200 U/L penicillin-streptomycin in a 37° C. incubator with about 5% CO₂ and passaged about 5-30 times prior to isolating.

Fetal bovine serum may harbor pathogens and isolated PVMC recipients may develop anti-FBS antibodies, requiring in some instances the use of serum-free media. In some embodiments, PVMCs can be grown in serum-free media supplemented with FGF, platelet-derived growth factor (PDGF) and transforming growth factor-β (TGF-β). In further embodiments, platelet-rich plasma also appears an effective substitute for FBS. Autologous serum also represents a viable alternative, though limited by the large volume necessary to supplement the media. In addition, several serum-free defined media are commercially available for the expansion for mesenchymal stem cells that will also be applicable to the expansion of PVMCs. In some embodiments, autologous serum can be used to supplement serum free media. In some embodiments, autologous serum is added to serum-free media to achieve a final concentration of about 5 to about 25%. In some embodiments, the autologous serum may be autologous human serum. The use of such chemically defined media requires that they be optimized for a particular source of PVMC and, in some cases, for a particular therapeutic use for PVMCs.

### Methods for Preparing Demineralized Bone

In some embodiments, PVMC preparations may be prepared using demineralized bone. In some embodiments, a method of preparing demineralized bone comprises mixing an acid with the bone material to make demineralized bone powder. In some embodiments, demineralized bone may include fully demineralized bone or partially demineralized bone. In some embodiments, bone material may include bone chips, bone powder, or a combination thereof. In some embodiments, a mixture of bone chips and bone powder may be used to make demineralized bone. In some embodiments, bone chips are used to make demineralized bone. In some embodiments, the method further comprises grinding bone to make bone chips. In some embodiments, the method further comprises milling bone chips to make bone powder. In some embodiments, acid may be a strong mineral acid. As used herein, bone chips may refer to bone fragments, bone segments, bone pieces, bone slivers or the like. In some embodiments, the acid may be selected from hydrochloric acid, nitric acid, phosphoric acid, sulfuric acid, boric acid, hydrofluoric acid, or hydrobromic acid. In some embodiments, mixing the bone powder with the acid may be for a period of about 6 hours to about 48 hours. In some embodiments, the period of mixing the bone powder with the acid may be about 6 to about 36 hours, about 6 to about 24 hours, about 12 to about 48 hours, about 12 to about 36 hours, about 12 to about 24 hours, or a range between any two of these values.

In some embodiments, the method further comprises soaking a bone material in ethanol. In some embodiments, the ethanol is 70% EtOH. In some embodiments, another sterilizing antibacterial or antifungal agent can be used. In some embodiments, the method may comprise grinding the bone to make bone chips. In some embodiments, the chips may be in the form of irregularly shaped polyhedra with an edge dimension up to about 5 mm. In some embodiments, chips may be in the form of irregularly shaped polyhedra with an edge dimension up to about 10 mm. In some embodiments, the method further comprises milling the bone chips and placing the chips in a sieve to size the milled bone to about 100-800 microns to make bone powder. In some embodiments, bone chips, from a proximal tibia, may be milled to form particles ranging in size from about 3.6 mm to about 8.0 mm. In some embodiments, bone chips from a distal femur may be milled to form particles ranging in size from about 2.0 mm to about 8.0 mm. In some embodiments, bone chips from a femoral head may be milled to form particles ranging in size from about 2.0 mm to about 5.0 mm. The size of a milled bone chip may be measured around its largest axis. In some embodiments, the milled bone material may be placed in a mixing container and cleaned with hydrogen peroxide and stirred. In some embodiments, the bone chips may be subject to rinsing without milling the bone. In some embodiments, the bone material may then be removed and rinsed with sterile water. In further embodiments, the rinsed bone material may be placed back into the cleaned mixing container and ethanol, or another sterilizing, antibacterial or antifungal agent may be added and the solution may be mixed. In some embodiments, the bone material may be transferred into a sieve and an open vacuum may be applied to the bottom of the sieve and the bone powder may be dried. In some embodiments, the dried bone material may be transferred to the partial demineralization process where it may be weighed. In further embodiments, the bone material may be mixed with acid. In some embodiments, the longer the bone material is left in contact with the acid, the greater the degree of demineralization of the bone powder.

In some embodiments, the bone material may be cleaned with a ratio of about 2:1, about 3:1, about 4:1, about 5:1, about 6:1, about 7:1, about 8:1, about 9:1 or about 10:1 of 3% aqueous Hydrogen Peroxide (H₂O₂) to bone and stirred for about 5 to 30 minutes. In some embodiments, the bone material may then be removed and rinsed with sterile water. Ethanol or another sterilizing, antibacterial or antifungal agent may be added to the rinsed bone material to make a solution. In some embodiments, the solution may be mixed for about 10 to about 60 minutes. In some embodiments, the method further comprises drying the solution to form a dried bone material. In some embodiments, the bone powder may be transferred into a No. 70 sieve and an open vacuum may be applied to the bottom of the sieve and the bone powder may be dried for about 10 to about 60 minutes. In some embodiments, the dried bone material may be weighed and the bone weight in grams may be compared to a chart which determines the acid volume to be applied. In some embodiments, the amount of acid to be added may be about 16 mL for every 1 gram of bone. In some embodiments, the bone material may be mixed with a strong mineral acid such as hydrochloric acid, nitric acid, phosphoric acid, sulfuric acid, boric acid, hydrofluoric acid, or hydrobromic acid for about 1 to about 12 hours to achieve partial bone to surface engagement with the hydrochloric acid in order to make partially demineralized bone. In further embodiments, the bone material may be mixed with a strong mineral acid such as hydrochloric acid, nitric acid, phosphoric acid, sulfuric acid, boric acid, hydrofluoric acid, or hydrobromic acid for about 12 to about 24 hours in order to achieve maximum bone surface engagement with the Hydrochloric acid to remove most of the mineral content. In some embodiments, the bone material may be mixed with a strong mineral acid for a longer period of time to fully demineralize the bone.

In some embodiments, partially demineralized bone chips or fully demineralized bone chips may be combined with isolated PVMCs to form a therapeutic preparation that may be useful in the promoting bone regeneration and creating a regenerative environment for tissue regeneration and repair.

### Methods for Making Bone Chips

Some embodiments are a method of producing bone chips comprising passing a bone fragment through a grinder or bone mill. In some embodiments, a bone chip comprises bone tissue, bone marrow, or a combination thereof. In some embodiments, a bone chip comprises compact bone, bone marrow, tissue from the medullary canal, cancellous tissue, or combinations thereof. In some embodiments, fragments of bone are milled to form bone chips. In some embodiments, the fragments of bone are milled by passage through a bone mill or grinder.

In some embodiments, the bone fragment originates from human bones comprising the femur, ilium crest, patella, tibia, humerus, clavicle, ribs or scapula.

In some embodiments, the bone fragment originates from the proximate femur, distal femur, or a combination thereof. In some embodiments, the bone fragment is fresh. In some embodiments, the bone fragment is cryogenically frozen. In some embodiments, the bone chips have an intact trabecular structure. In some embodiments, the bone chips comprise PVMCs. In some embodiments, bone chips comprise bone fragments with attached marrow tissue and other tissue.

Bone chips can vary in size depending on their origin. In some embodiments, a bone fragment is milled to form a bone chip ranging in size from 3.6 mm to 8.0 mm. In some embodiments, a bone fragment originating from a distal femur is milled to form a bone chip ranging in size from about 2.9 mm to about 7.1 mm. In some embodiments, a bone fragment origination from a femoral head may be milled to form particles ranging in size from about 2.2 mm to about 3.4 mm. In some embodiments, the size of a milled bone chip is measured around its largest axis.

In some embodiments, a method of producing bone chips comprises passing a bone fragment through a grinder or bone mill further comprises cryogenically preserving a bone chip.

In some embodiments, a method of producing bone chips comprises passing a bone fragment through a grinder or bone mill further comprises therapeutic administration to a patient in need thereof.

### Methods for Isolating Osteogenic Cells

In some embodiments, osteogenic cells may be mixed with PVMC preparations, bone chips, bone powder, or combinations thereof, and contribute to creating a regenerative microenvironment for bone.

In some embodiments, osteogenic cells can be isolated from a PVMC preparation. Osteogenic cells may be identified within a population of PVMCs due to high levels of expression of alkaline phosphatase (AP). Antibodies to AP are commercially available and can be conjugated to fluorescent tags to enable sorting of cells expressing high levels of AP (AP-High cells).

In some embodiments, combinations of PVMC preparations and bone marrow cells can be cultured in media with dexamethylsome at a final concentration of about 1 nM to about 100 nM and ascorbate or ascorbate-2-phosphate for periods of 5 to 10 days. In some embodiments, cells are cultured for 7 days. In further embodiments, the medium used serves as an induction medium for the growth of osteogenic cells. PVMCs are adherent cells and can be disaggregated and released from the culture dish following trypsin and collagenase digestion. Disaggregated cells can then be sorted by AP expression levels allowing for isolation of AP-High cells. The AP-High cells may be segregated and may serve as a standard for the intrinsic osteogenic cells in fresh preparations. Furthermore, the AP-High cells may be used to stain osteogenic cells in vivo and in vitro.

Some embodiments are directed to a method for separating osteogenic cells from a PVMC preparation comprising mixing the PVMC preparation with an antibody having an affinity to high-specific activity alkaline phosphatase.

Some embodiments are directed to a method for separating osteogenic cells from a PVMC preparation comprising determining adsorption of a cell in the preparation to calcium phosphate substrates, wherein a high affinity indicates the presence of the osteogenic cell.

Some embodiments are directed to a method of separating osteogenic cells from a PVMC preparation comprising plating the PVMC preparation onto a coated petri dish and isolating osteogenic cells based on the differential attachment of the cells. In some embodiments, the coated petri dish is coated with fibronectin, laminin, other extracellular matrix molecules, or a combination thereof.

### Methods for Making an Enhanced Autologous Bone Graft

Some embodiments may be directed to a method of making an enhanced, autologous bone graft comprising:
a. extracting a cell suspension from a first portion of bone tissue from a subject with an enzyme, mechanical force, or a combination thereof;
b. concentrating the cells in the cell suspension by buoyant density sedimentation, filtration or centrifugation to obtain a population of concentrated bone-derived PVMCs; and
c. supplementing a second portion of bone tissue or bone substitute to be used as a bone graft from the subject with the population of concentrated bone-derived PVMCs, so as to make the enhanced, autologous bone graft.

In some embodiments, a method of making an enhanced, autologous bone graft comprises isolating bone-derived PVMCs from a subject and supplementing a bone portion from the subject to be used as a bone graft with the bone-derived PVMCs. In some embodiments, a method of making an enhanced, autologous bone graft comprises isolating bone-derived PVMCs from a first portion of bone tissue of a subject, extracting a second portion of bone tissue from the subject to be used as a bone graft and supplementing the bone graft with the bone-derived PVMCs.

In some embodiments, extracting a cell suspension from a first portion of bone tissue comprises enzymatic digestion to specifically cleave bonds joining the perivascular cells to a basement membrane of small blood vessels in the bone. More specifically, enzymatic digestion can cleave molecules that may be part of the basement membrane, releasing PVMC binding domains as a result.

In some embodiments, enhanced, autologous bone grafts, containing PVMCs can be produced by extracting from a subject a first portion of bone tissue to be used as a bone graft then supplementing the bone graft with a population of concentrated bone-derived PVMCs, wherein the concentrated bone-derived PVMCs may be prepared by extracting and concentrating the PVMCs from a second portion of bone tissue from the subject, so as to make the enhanced, autologous bone graft.

In some embodiments, a first portion of bone tissue originates from bone. In some embodiments, a second portion of bone tissue originates from bone, bone chips, bone marrow, tissue from a bone cavity lavage, or combinations thereof. In some embodiments, a first portion of bone tissue comprises bone marrow and a second portion of bone tissue comprises bone chips.

In some embodiments, the first portion of bone tissue originates from human bones comprising ilium crest, femur, patella, tibia, humerus, clavicle, ribs or scapula, or combinations thereof. In some embodiments, this tissue may be obtained as discarded tissue following surgical operations on patients and prepared for autologous use. In some embodiments, bone can originate from the proximate and distal regions of the femur, ilium crest, patella, tibia, humerus, clavicle, ribs, scapula, or combinations thereof.

In another embodiment, the second portion of bone tissue originates from human bones comprising ilium crest, femur, patella, tibia, humerus, clavicle, ribs or scapula, or combinations thereof.

In some embodiments, the enhanced autologous bone graft may be supplemented by PVMCs isolated from bone. In some embodiments, undemineralized, demineralized bone chips or partially demineralized bone chips may be used in combination with bone powder or with bone derived PVMCs to make an enhanced autologous bone graft preparation.

In some embodiments, the method of making an enhanced, autologous bone graft can include the step of supplementing an enhanced, autologous bone graft with fresh autologous bone marrow, processed autologous bone marrow, frozen autologous bone marrow, or combinations thereof.

In some embodiments, the method of making an enhanced, autologous bone graft comprises supplementing an enhanced, autologous bone graft with addition of one or more synthetic bone substitutes, wherein the synthetic bone substitutes comprise a calcium phosphate-based bone substitute, calcium apatite, β-tricalcium phosphate, natural and synthetic polymers, ceramics, Allogro, Opteform, Grafton, OrthoBlast, calcium phosphate, calcium sulfate, bioglass, OsteoGraf, Norian SRS, ProOsteon, Osteoset, polymer-based bone graft substitutes, degradable and nondegradable polymers, Cortoss, open porosity polylactic acid polymer Immix, or combinations thereof.

In some embodiments, the method of making an enhanced, autologous bone graft can comprise adding mineralized processed allograft, minimally demineralized processed allograft, partially demineralized processed allograft, demineralized processed allograft, or a combination thereof.

In some embodiments, the method of making an enhanced, autologous bone graft comprises the addition of collagen sponge, BMP-2-containing collagen sponge, BMP-7-containing collagen sponge, BMP-2 and BMP-7 containing sponge, or combinations thereof. In some embodiments, the method of making an enhanced, autologous bone graft can include the addition of PDGF-BB. In some embodiments, the method of making an enhanced, autologous bone graft comprises adding PDGF-BB to a collagen sponge or another suitable vehicle. In some embodiments, the method of making an enhanced, autologous bone graft comprises the addition of PDGF-BB-containing collagen sponge

In some embodiments, the autologous bone graft may comprise a calcium phosphate-based bone substitute combined with isolated osteoblasts, whole marrow, unpurified, purified or expanded PVMCs, isolated PVMCs, or combinations thereof. In some embodiments, autologous bone fragments can be combined with other bone substitutes such as hydroxyapatite, calcium apatite, and β-tricalcium phosphate. In some embodiments, the autologous bone graft comprises natural and synthetic polymers, ceramics, or other bone substitute materials, or combinations thereof in addition to comprising isolated osteoblasts, whole marrow, unpurified, purified or expanded PVMCs, isolated PVMCs or a combination thereof. In further embodiments, the bone may be from discarded knee or hip bone/marrow obtained during route arthroplasty.

In some embodiments, autologous bone graft may comprise a PVMC isolated from a periosteum, trabecular bone, adipose tissue, synovium, skeletal muscle, deciduous teeth, pancreas, lung, liver, amniotic fluid, placenta, blood and umbilical cord blood vessel, or combinations thereof. In some embodiments, autologous bone graft may comprise a PVMC isolated from an umbilical cord blood vessel.

In some embodiments, the PVMCs may be obtained from a trabecular bone cavity of the bone. In some embodiments, PVMCs may be obtained from the femoral head, the distal femur or proximal tibia. In some embodiments, the bone comprises bone marrow tissue and other tissue, compact bone, bone marrow from the medullary canal, or combinations thereof.

In further embodiments, autologous bone graft substitutes can be used alone or in combination with other materials (e.g., Allogro, Opteform, Grafton, or OrthoBlast). In some embodiments, ceramic-based bone graft substitutes including calcium phosphate, calcium sulfate, and bioglass can be used alone or in combination (e.g., OsteoGraf, Norian SRS, ProOsteon, or Osteoset). In some embodiments, polymer-based bone graft substitutes, degradable and nondegradable polymers, may be used alone or in combination with other materials (e.g., Cortoss, open porosity polylactic acid polymer, or Immix).

In some embodiments, an enhanced autologous graft can also comprise processed allograft bone material, for example, mineralized processed allograft, minimally demineralized processed allograft, partially demineralized processed allograft, or demineralized processed allograft. In some embodiments, an enhanced autologous graft can also comprise a collagen sponge, a BMP-2-containing collagen sponge, a BMP-7-containing collagen sponge, a BMP-2 and BMP-7 containing sponge, or combinations thereof.

Some embodiments comprise a method of making an enhanced, autologous bone graft comprising extracting from a subject a first portion of bone tissue to be used as a bone graft then supplementing the bone graft with a population of concentrated bone-derived PVMCs, wherein the concentrated bone-derived PVMCs may be prepared by extracting and concentrating the PVMCs from a second portion of the same autologous bone tissue from the subject, so as to make the enhanced, autologous bone graft.

In some embodiments, the method of making an enhanced, autologous bone graft can include the steps of adding a calcium phosphate-based bone substitute, isolated osteoblasts, whole marrow, unpurified, purified or expanded PVMCs, or combinations thereof. In some embodiments, the method of making an enhanced, autologous bone graft can include the steps of adding autologous bone fragments, bone substitutes such as but not limited to hydroxyapatite, calcium apatite, β-tricalcium phosphate, or combinations thereof. In some embodiments, the autologous bone graft comprises natural and synthetic polymers, ceramics, or other bone substitute materials, or combinations thereof, in addition to comprising isolated osteoblasts, whole marrow, unpurified, purified or expanded PVMCs.

In some embodiments, the method of making an enhanced, autologous bone graft can include the steps of adding autologous bone graft substitutes alone or in combination with other materials (e.g., Allogro, Opteform, Grafton, or OrthoBlast). In some embodiments, ceramic-based bone graft substitutes including calcium phosphate, calcium sulfate, and bioglass can be added alone or in combination (e.g., OsteoGraf, Norian SRS, ProOsteon, or Osteoset). In some embodiments, polymer-based bone graft substitutes, degradable and nondegradable polymers may be added alone or in combination with other materials (e.g., Cortoss, open porosity polylactic acid polymer, or Immix).

In some embodiments, a method of making an enhanced, autologous graft can also comprise the step of adding processed allograft bone material, for example, mineralized processed allograft, minimally demineralized processed allograft, partially demineralized processed allograft, or demineralized processed allograft. In some embodiments, a method of making an enhanced, autologous graft can also comprise the step of adding a collagen sponge, a BMP-2-containing collagen sponge, a BMP-7-containing collagen sponge, a BMP-2 and BMP-7 containing sponge, or combinations thereof.

In another embodiment, a method of making an enhanced, autologous bone graft additionally comprises culturing the concentrated PVMCs before step (c) to selectively expand the portion of the PVMC portion of the population of concentrated bone-derived PVMCs. In another embodiment, the population of concentrated bone-derived PVMC may not be cultured.

In some embodiments, PVMC preparations and autologous bone grafts containing PVMCs can be used to stimulate bone regeneration by administering a composition comprising a therapeutically effective amount of PVMCs either with or without bone marrow to the torso, head or limbs of a human patient. In some embodiments, the administered perivascular cells may be capable of directly differentiating into secretory osteoblasts and/or providing a regenerative microenvironment for bone formation.

### PVMC Preparations and Administration

In some embodiments, the PVMCs may be for autologous use. In some embodiments, PVMCs may be isolated from a subject and these cells may be administered back to the subject from whom they were raised. In some embodiments, autologous transfer may prevent the need for immunosuppressive protocols.

In some embodiments, isolated PVMCs can be utilized therapeutically. In some embodiments, isolated PVMCs can form part of an allogeneic infusate. In some embodiments, isolated PVMCs can be combined with isolated osteoblasts, whole marrow, unpurified, purified or expanded PVMCs to form an infusate. In some embodiments, an infusate can also include a balanced salt solution comprising phosphate buffered saline, lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), or a combination thereof. In some embodiments, isolated PVMCs can be injected directly into a tissue. For example, a preparation of isolated PVMCs can be injected directly into the heart via a catheter. In some embodiments, isolated PVMCs can be encased in a scaffold prior to therapeutic administration. Examples of suitable scaffolds include preformed struts and crosslinking complexes activated by an enzyme or catalyst such that cross linking occurs in vivo.

In some embodiments, isolated PVMC preparations may be administered to the torso, head or limbs of a human patient and may be capable of providing a regenerative microenvironment for bone regeneration. In some embodiments, the intrinsic secretory activity of isolated PVMCs may establish a regenerative microenvironment at sites of tissue injury to damage. In some embodiments, isolated PVMCs may secrete bioactive factors that inhibit scarring, inhibit apoptosis, stimulate angiogenesis and stimulate the mitosis and tissue-intrinsic stem or progenitor cells and also secrete antibiotic proteins when bacteria may be present at the site of injury, for example, an open wound. The multifaceted effects of isolated PVMCs can be referred to as “trophic activity”.

In some embodiments, the medicinal capabilities of the isolated PVMCs may be defined by the spectrum of molecules secreted by the PVMCs in a particular physiological environment as determined by specific, different anatomic locations. In some embodiments, the medicinal capabilities of the PVMCs are defined by the spectrum of molecules secreted by the PVMC. In some embodiments, the spectrum of molecules secreted by the PVMC is site-dependent.

In some embodiments, PVMCs isolated from human bone tissue can be used in autologous grafts and may provide a regenerative environment via the secretion of prostaglandin E2 (PGE2), stromal-cell derived factor-1 (SDF-1), Vascular endothelial growth factor (VEGF), interleukin-7 (IL-7) and interleukin-8 (IL-8).

In some embodiments, isolated PVMCs may provide an anti-apoptotic microenvironment via the secretion of vascular endothelial growth factor (VEGF), hepatocyte growth factor (HGF) transforming growth factor beta (TGF-β), basic fibroblast growth factor (bFGF) and granulocyte-macrophage colony-stimulating factor (GM-CSF) and insulin-like growth factor 1 (IGF-1), factors that enhance endothelial cell growth and survival.

In some embodiments, isolated PVMCs may have immunomodulatory properties via inhibition of the proliferation of αβ T-cells, suppression of γδ T-cells, inhibition and promotion of B cell proliferation, suppression of NK cell activation, modulation of the cytokine secretion profile of dendritic cells and macrophages and suppression of immunoglobulin production by plasma cells. Prostaglandin E2 (PGE-2) may be a central mediator in many of the effects of PVMCs on immune cells, and in the modulation of the secretory profile of dendritic cells and macrophages. TGF-β1 and HGF secreted by PVMCs may have an immunomodulatory role. PVMCs may also express indoleamine 2,3-dioxygenase (IDO) which may halt T-cell proliferation. Other molecules that mediate immunomodulatory effects of PVMCs may include interleukin (IL)-10, human leukocyte antigen G (HLA-G) and leukemia inhibitory factor (LIF), the latter playing an important role not only in the suppression of T-cell proliferation, but also in the generation and maintenance of regulatory T-cells. In yet another embodiment, PVMCs may be able to inhibit pro-inflammatory cytokines interleukin-1β (IL-1β), interleukin-2 (IL-2) interferon-γ (IFN-γ), tumor necrosis factor-α (TNF-α), and interleukin-1α (IL-1α). In yet another embodiment, PVMCs, under the influence of low doses of IFN-γ, may express class II major histocompatibility complex (MHC) molecules and behave as antigen-presenting cells.

In some embodiments, isolated PVMCs may also play a role during tissue repair. PVMCs may be able to secrete different bioactive molecules that act in concert to resolve the lesion. Without wishing to be bound by theory, it is likely that in the early steps of the process, PVMCs may provide a supportive effect on immune cells via expression of pro-inflammatory molecules that may be chemoattractant to inflammatory cells namely eotaxin, granulocyte colony stimulating factor (G-CSF) and IL-8 and regulated upon activation, normal T-cell expressed and secreted (RANTES). Exposure of PVMCs to TNF-α or IL-1β may increase the expression of chemoattractive and stimulatory molecules including IL-1β, IL-6, IL-7, IL-12, IL-16, IL-1 receptor antagonist (IL-1ra), TNF-α, tumor necrosis factor-β (TNF-β), epithelial neutrophil-activating protein 78 (ENA-78), eotaxin, IL-8, monocyte chemoattractant protein 1 (MCP-1), macrophage inflammatory protein-1α (MIP-1 α), MIP-1β, RANTES, intercellular adhesion molecule-1 (ICAM-1), VCAM-1, G-CSF, GM-CSF, growth hormone, stem cell factor (SCF), VEGF₁₆₅, bFGF, thyroid-stimulating hormone (TSH), CD40, and CD40 ligand. In some embodiments, PVMCs may be able to respond to inflammatory cells at the early stages of wound healing and provide physiological support for the subsequent steps of the immune response. However, it may be that as the local environment undergoes changes during the healing process, expression profile of PVMCs changes with time, thus resulting in inhibition of the immunosurveillance of the injury site and prevention the initiation of autoimmune events.

In some embodiments, isolated PVMCs may have an anti-fibrotic effect before the establishment of massive fibrosis takes place. HGF and bFGF may be involved in the prevention of fibrosis by PVMCs. In a situation of tissue injury, PVMCs may become proliferative and secrete HGF, which in turn mediates anti-fibrotic and immunomodulatory effects. In some embodiments, administration of PVMCs to prevent fibrosis can, thus, be viewed as a way to augment local production of HGF (and probably other anti-scarring factors) in cases where fibrosis is to be avoided.

In some embodiments, isolated PVMCs may be able to support hematopoiesis in vitro, and this ability may involve the constitutive secretion of soluble factors such as SCF, LIF, IL-6, and macrophage colony-stimulating factor (M-CSF). In addition, hematopoietic support can be further augmented by IL-1α-induced secretion of G-CSF and GM-CSF.

Without wishing to be bound by theory, it is believed that establishment of blood supply is fundamental for recovery of damaged tissues. In some embodiments, PVMCs may have a pro-angiogenic effect via the secretion of bFGF, VEGF, placental growth factor (PlGF), and MCP-1 as well as angiogenic and anti-apoptotic factors such as IL-6, VEGF and MCP-1, which inhibit the death of endothelial cells cultured under hypoxic conditions and promote the formation of capillary-like structures in in vitro assays. PVMCs may also be able to contribute to angiogenesis by providing extracellular matrix components that serve as a substrate for endothelial cells. In some embodiments, PVMCs may transition into pericytes and stabilize the newly formed vasculature.

In some embodiments, isolated PVMCs may secrete a variety of chemoattractant molecules, which include CCL2 (MCP-1), CCL3 (MIP-1α), CCL4 (MIP-1β), CCL5 (RANTES), CCL7 (MCP-3), CCL20 (MIP-3α), CCL26 (eotaxin-3), CX3CL1 (fractalkine), CXCL5 (ENA-78), CXCL11 (i-TAC), CXCL1 (GROα), CXCL12 (SDF-1), CXCL8 (IL-8), CXCL2 (GROβ), and CXCL10 (IP-10). Target cells for these chemoattractant molecules include monocytes, eosinophils, neutrophils, basophils, memory and naïve T-cells, B cells, NK cells, dendritic cells, and hematopoietic and endothelial progenitors. In some embodiments, chemokine expression by PVMCs may be modified by exposure to other cell types, particularly immune cells.

In some embodiments, compositions comprising pure, substantially pure, or impure isolated PVMCs, have medicinal capabilities. In further embodiments, the PVMCs of these compositions have medicinal capabilities. In some embodiments, the PVMCs may be capable of expressing CD146, CD105, CD166, CD44, CD73, CD90, or a combination thereof. In some embodiments, the PVMCs may be CD45 negative. In some embodiments, compositions comprise PVMCs isolated from bone and bone marrow cells.

In some embodiments, pure, substantially pure or impure PVMC preparations may be administered to the torso, head, or limbs of a human patient. In yet another embodiment, PVMCs may be capable of providing a regenerative microenvironment.

In some embodiments, pure, substantially pure or impure PVMC preparations can be combined with bone chips, fully demineralized bone chips, or partially demineralized bone chips.

Some embodiments are directed to a pharmaceutical composition comprising a therapeutically effective amount of a plurality of isolated PVMCs and a pharmaceutically acceptable carrier. In some embodiments, the plurality of PVMCs comprises PVMCs derived from bone, umbilical cord blood vessel, an anatomic source containing PVMCs, or a combination thereof. In some embodiments, the bone comprises a bone chip, a trabecular bone cavity, bone marrow, bone cavity lavage, or a combination thereof. In some embodiments, the PVMCs may be derived from any anatomic sources that may contain PVMCs. Examples of anatomic sources include but are not limited to blood vessels including, but limited to veins and arteries, periosteum, trabecular bone, adipose tissue, synovium, skeletal muscle, deciduous teeth, pancreas, lung, liver, amniotic fluid, placenta, blood. In some embodiments, the PVMC express a CD selected from CD146, CD105, CD166, CD44, CD73, CD90, or a combination thereof. In some embodiments, the PVMC does not express CD45. Some embodiments further comprise bone marrow cells. Some embodiments further comprise a scaffold material. In some embodiments, the scaffold material comprises bone chips, ceramic-based bone graft substitutes, calcium phosphate ceramics, calcium sulfate ceramics, bioglass, polymer-based bone graft substitutes, degradable and nondegradable polymers, processed allograft bone material, mineralized processed allograft, demineralized processed allograft, collagen sponges, or combinations thereof.

Some embodiments are directed to an isolated PVMC. In some embodiments, the PVMC is derived from bone. In some embodiments, the bone comprises a bone chip, a trabecular bone cavity, bone marrow, bone cavity lavage, or a combination thereof. In some embodiments, the PVMC is derived from an umbilical cord blood vessel.

Some embodiments are directed to a composition comprising a plurality of PVMCs and an acceptable carrier. In some embodiments, the plurality of PVMCs comprises PVMCs derived from bone, an umbilical cord blood vessel or a combination thereof. In some embodiments, the bone comprises bone chips, bone marrow tissue and other tissue, compact bone, bone marrow from an intermedullary canal, a bone chip, a trabecular bone cavity, a bone cavity lavage, or combinations thereof. In some embodiments, the PVMC expresses a CD selected from CD146, CD105, CD166, CD44, CD73, CD90, and a combination thereof. In some embodiments, the PVMC does not express CD45. Some embodiments further comprise bone marrow cells. Some embodiments further comprise a scaffold material. In some embodiments, the scaffold material comprises bone chips, ceramic-based bone graft substitutes, calcium phosphate ceramics, calcium sulfate ceramics, bioglass, polymer-based bone graft substitutes, degradable and nondegradable polymers, processed allograft bone material, mineralized processed allograft, demineralized processed allograft, collagen sponges, or combinations thereof.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins which is hereby incorporated by reference in its entirety.

### Therapeutic Use of PVMCs

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature.

The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician.

Some embodiments are directed towards a method of treating a patient in need thereof, comprising administering an allogeneic infusate. In some embodiments, the infusate comprises a balanced salt solution, isolated PVMCS, or a combination thereof.

In some embodiments, isolated PVMCs can be utilized therapeutically. In some embodiments, PVMCs isolated from an umbilical cord blood vessel can form part of an allogeneic infusate. In some embodiments, PVMCs isolated from bone can form part of an allogeneic infusate. In some embodiments, isolated PVMCs isolated can be combined with isolated osteoblasts, whole marrow, unpurified, purified or expanded PVMCs to form an infusate. In some embodiments, an infusate can also include a balanced salt solution comprising phosphate buffered saline (PBS), lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), or a combination thereof. In some embodiments, PVMCs isolated from an umbilical cord blood vessel can be injected directly into a tissue. In some embodiments, isolated PVMCs can be encased in a scaffold prior to therapeutic administration. Examples of suitable scaffolds include preformed struts and cross linking complexes activated by an enzyme or catalyst such that cross linking occurs in vivo.

In some embodiments, Isolated PVMC preparations may be administered to the torso, head or limbs of a human patient and may be capable of providing a regenerative microenvironment for tissue regeneration. In some embodiments, the intrinsic secretory activity of isolated PVMCs establishes a regenerative microenvironment at sites of tissue injury to damage. In some embodiments, isolated PVMCs may secrete bioactive factors that inhibit scarring, inhibit apoptosis, stimulate angiogenesis and stimulate the mitosis and tissue-intrinsic stem or progenitor cells and secrete antibiotic proteins when bacteria is present at the site of injury, for example, an open wound. As used herein, the multifaceted effects of PVMCs may be referred to as “trophic activity”.

In some embodiments, isolated PVMCs can be used in allografts and are expected to provide a regenerative environment via the secretion of prostaglandin E2 (PGE2), stromal-cell derived factor-1 (SDF-1), Vascular endothelial growth factor (VEGF), interleukin-7 (IL-7) and interleukin-8 (IL-8).

In some embodiments, isolated PVMCs provide an anti-apoptotic microenvironment via the secretion of vascular endothelial growth factor (VEGF), hepatocyte growth factor (HGF), transforming growth factor beta (TGF-β), basic fibroblast growth factor (bFGF) and granulocyte-macrophage colony-stimulating factor (GM-CSF) and insulin-like growth factor 1 (IGF-1), factors that enhance endothelial cell growth and survival.

In some embodiments, isolated PVMCs may have immunomodulatory properties via inhibition of the proliferation of αβ T-cells, suppression of γδ T-cells, inhibition and promotion of B cell proliferation, suppression of NK cell activation, modulation of the cytokine secretion profile of dendritic cells and macrophages and suppression of immunoglobulin production by plasma cells. Prostaglandin E2 (PGE-2) may be a central mediator in many of the effects of PVMCs on immune cells, and in the modulation of the secretory profile of dendritic cells and macrophages. TGF-β1 and HGF, secreted by PVMCs, are also expected to have an immunomodulatory role. PVMCs may express indoleamine 2,3-dioxygenase (IDO) which has been shown to halt T-cell proliferation. Other molecules that mediate immunomodulatory effects of PVMCs may include interleukin (IL)-10, human leukocyte antigen G (HLA-G) and leukemia inhibitory factor (LIF) the latter playing an important role not only in the suppression of T-cell proliferation, but also in the generation and maintenance of regulatory T-cells. In some embodiments, PVMCs may inhibit pro-inflammatory cytokines interleukin-1β (IL-1β), interleukin-2 (IL-2) interferon-γ (IFN-γ), tumor necrosis factor-α (TNF-α), and interleukin-1α (IL-1α). In some embodiments, PVMCs, under the influence of low doses of IFN-γ, express class II major histocompatibility complex (MHC) molecules and behave as antigen-presenting cells.

In some embodiments, isolated PVMCs may also play a role during tissue repair. PVMCs may be able to secrete different bioactive molecules that act in concert to resolve the lesion. In some embodiments, during the early steps of the process, PVMCs may provide a supportive effect on immune cells via expression of pro-inflammatory molecules that are chemoattractant to inflammatory cells namely eotaxin, granulocyte colony stimulating factor (G-CSF) and IL-8 and regulated upon activation, normal T-cell expressed and secreted (RANTES). Exposure of PVMCs to TNF-α or IL-1β may result in the increased expression of chemoattractive and stimulatory molecules including IL-1β, IL-6, IL-7, IL-12, IL-16, IL-1 receptor antagonist (IL-1ra), TNF-α, tumor necrosis factor-β (TNF-β), epithelial neutrophil-activating protein 78 (ENA-78), eotaxin, IL-8, monocyte chemoattractant protein 1 (MCP-1), macrophage inflammatory protein-1α (MIP-1α), MIP-1 β, RANTES, intercellular adhesion molecule-1 (ICAM-1), VCAM-1, G-CSF, GM-CSF, growth hormone, stem cell factor (SCF), VEGF₁₆₅, bFGF, thyroid-stimulating hormone (TSH), CD40 and CD40 ligand. PVMCs may be able to respond to inflammatory cells at the early stages of wound healing and provide physiological support for the subsequent steps of the immune response. However, as the local environment undergoes changes during the healing process, the expression profile of PVMCs may change with time resulting in inhibition of the immunosurveillance of the injury site and prevent the initiation of autoimmune events.

In some embodiments, isolated PVMCs may have an anti-fibrotic effect before the establishment of massive fibrosis takes place. bFGF and HGF may be involved in the prevention of fibrosis by PVMCs. In a situation of tissue injury, PVMCs may become proliferative and secrete HGF, which in turn, mediates anti-fibrotic and immunomodulatory effects. In some embodiments, administration of PVMCs to prevent fibrosis can, thus, be viewed as a way to augment local production of HGF (and probably other anti-scarring factors) in cases where fibrosis is to be avoided.

In some embodiments, isolated PVMCs may be able to support hematopoiesis in vitro and this ability may involve the constitutive secretion of soluble factors such as SCF, LIF, IL-6 and macrophage colony-stimulating factor (M-CSF); in addition, hematopoietic support can be further augmented by IL-1α-induced secretion of G-CSF and GM-CSF.

Establishment of blood supply may be fundamental for recovery of damaged tissues. In some embodiments, Isolated PVMCs may have a pro-angiogenic effect via the secretion of bFGF, VEGF, placental growth factor (PlGF), and MCP-1 as well as angiogenic and anti-apoptotic factors such as IL-6, VEGF and MCP-1, which inhibit the death of endothelial cells cultured under hypoxic conditions and promote the formation of capillary-like structures in vitro assays. In some embodiments, isolated PVMCs are also expected to be able to contribute to angiogenesis by providing extracellular matrix components that serve as a substrate for endothelial cells. It is also expected that PVMCs may transition into pericytes and stabilize the newly formed vasculature.

In some embodiments, isolated PVMCs may secrete a variety of chemoattractant molecules, which include CCL2 (MCP-1), CCL3 (MIP-1α), CCL4 (MIP-1β), CCL5 (RANTES), CCL7 (MCP-3), CCL20 (MIP-3α), CCL26 (eotaxin-3), CX3CL1 (fractalkine), CXCL5 (ENA-78), CXCL11 (i-TAC), CXCL1 (GROα), CXCL12 (SDF-1), CXCL8 (IL-8), CXCL2 (GROβ) and CXCL10 (IP-10). Target cells for these include monocytes, eosinophils, neutrophils, basophils, memory and naïve T-cells, B cells, NK cells, dendritic cells and hematopoietic and endothelial progenitors. It is likely that chemokine expression by PVMCs will be modified by exposure to other cell types, particularly immune cells.

In some embodiments, isolated PVMCs, PVMC preparations or a combination thereof, can be used to stimulate bone regeneration by administering a composition comprising a therapeutically effective amount of isolated PVMCs. In some embodiments, the isolated PVMCs may further comprise bone marrow. In some embodiments, administering the composition is to the torso, head or limbs of a human patient. The administered perivascular cells may be capable of directly differentiating into secretory osteoblasts and/or providing a regenerative microenvironment for bone formation.

Isolated PVMC preparations may be administered to the torso, head or limbs of a human patient and may be capable of providing a regenerative microenvironment for bone regeneration. The intrinsic secretory activity of isolated PVMCs establishes a regenerative microenvironment at sites of tissue injury to damage. As used herein the term “trophic activity” refers to the isolated PVMCs' ability to secrete bioactive factors that inhibit scarring, inhibit apoptosis, stimulate angiogenesis and stimulate the mitosis and tissue-intrinsic stem or progenitor cells and also secrete antibiotic proteins when bacteria is present at the site of injury, for example, an open wound.

In some embodiments, isolated PVMCs may provide a regenerative environment via the secretion of prostaglandin E2 (PGE2), stromal-cell derived factor-1 (SDF-1), Vascular endothelial growth factor (VEGF), interleukin-7 (IL-7) and interleukin-8 (IL-8).

In some embodiments, the isolated PVMCs may provide an anti-apoptotic microenvironment via the secretion of vascular endothelial growth factor (VEGF), hepatocyte growth factor (HGF) transforming growth factor beta (TGF-β), basic fibroblast growth factor (bFGF) and granulocyte-macrophage colony-stimulating factor (GM-CSF) and insulin-like growth factor 1 (IGF-1), factors that enhance endothelial cell growth and survival.

In some embodiments, isolated PVMCs may have anti-apoptotic effects in ischemic tissues. In some embodiments, isolated PVMCs may secrete molecules that protect against cell death caused by broken or malfunctioning blood vessels that result in decreased oxygen and nutrient supply to damaged tissues.

In some embodiments, isolated PVMCs may have immunomodulatory properties via inhibition of the proliferation of αβ T-cells, suppression of γδ T-cells, inhibition and promotion of B cell proliferation, suppression of NK cell activation, modulation of the cytokine secretion profile of dendritic cells and macrophages and suppression of immunoglobulin production by plasma cells. Prostaglandin E2 (PGE-2) may be a central mediator in many of the effects of PVMCs on immune cells and in the modulation of the secretory profile of dendritic cells and macrophages. TGF-β1 and HGF, secreted by PVMCs, may have an immunomodulatory role. PVMCs may express indoleamine 2,3-dioxygenase (IDO) which has been shown to halt T-cell proliferation. In some embodiments, other molecules that mediate immunomodulatory effects of isolated PVMCs may include interleukin (IL)-10, human leukocyte antigen G (HLA-G) and leukemia inhibitory factor (LIF) the latter playing an important role not only in the suppression of T-cell proliferation, but also in the generation and maintenance of regulatory T-cells. In some embodiments, isolated PVMCs may be able to inhibit pro-inflammatory cytokines interleukin-1β (IL-1β), interleukin-2 (IL-2) interferon-γ (IFN-γ), tumor necrosis factor-α (TNF-α), and interleukin-1α (IL-1α). In some embodiments, isolated PVMCs, under the influence of low doses of IFN-γ, express class II major histocompatibility complex (MHC) molecules and behave as antigen-presenting cells.

In some embodiments, isolated PVMCs may also play a role during tissue repair. In some embodiments, isolated PVMCs may be able to secrete different bioactive molecules that act in concert to resolve the lesion. It is likely that in the early steps of the process, PVMCs provide a supportive effect on immune cells via expression of pro-inflammatory molecules that are chemoattractant to inflammatory cells namely eotaxin, granulocyte colony stimulating factor (G-csf) and IL-8 and regulated upon activation, normal T-cell expressed and secreted (RANTES). In some embodiments, exposure of isolated PVMCs to TNF-α or IL-1β may result in the increased expression of chemoattractive and stimulatory molecules including IL-1β, IL-6, IL-7, IL-12, IL-16, IL-1 receptor antagonist (IL-1ra), TNF-α, tumor necrosis factor-β (TNF-β), epithelial neutrophil-activating protein 78 (ENA-78), eotaxin, IL-8, monocyte chemoattractant protein 1 (MCP-1), macrophage inflammatory protein-1α (MIP-1α), MIP-1β, RANTES, intercellular adhesion molecule-1 (ICAM-1), VCAM-1, G-CSF, GM-CSF, growth hormone, stem cell factor (SCF), VEGF₁₆₅, bFGF, thyroid-stimulating hormone (TSH), CD40, and CD40 ligand. In some embodiments, isolated PVMCs may be able to respond to inflammatory cells at the early stages of wound healing and provide physiological support for the subsequent steps of the immune response. In some embodiments, as the local environment undergoes changes during the healing process, it is likely that the expression profile of isolated PVMCs changes with time resulting in inhibition of the immunosurveillance of the injury site and prevention the initiation of autoimmune events.

In some embodiments, isolated PVMCs may have an anti-fibrotic or anti-scarring effect before the establishment of massive fibrosis takes place. HGF and bFGF may be involved in the prevention of fibrosis by isolated PVMCs. In a situation of tissue injury, isolated PVMCs may become proliferative and secrete HGF, which, in turn, mediates anti-fibrotic and immunomodulatory effects. In some embodiments, administration of isolated PVMCs to prevent fibrosis can, thus, be viewed as a way to augment local production of HGF (and probably other anti-scarring factors) in cases where fibrosis is to be avoided. In some embodiments, the anti-fibrotic or anti-scarring effects of isolated PVMCs may inhibit the entrance or function of myofibroblasts that move to the site of injury and normally fabricate dense collagenase scar tissue. In some embodiments, isolated PVMCs may be able to support hematopoiesis, and this ability may involve the constitutive secretion of soluble factors such as SCF, LIF, IL-6, and macrophage colony-stimulating factor (M-CSF); in addition, hematopoietic support can be further augmented by IL-1α-induced secretion of G-CSF and GM-CSF.

In some embodiments, isolated PVMCs may have mitogenic properties by secreting mitogens that stimulate tissue intrinsic progenitors to divide and differentiate resulting in regeneration of tissue at the site of injury.

In some embodiments, isolated PVMCs administered to a subject in need thereof may have angiogenic effects. Molecules secreted by isolated PVMCs may result in the recruitment of vascular endothelial cells or their progenitors to a site of injury. Once recruited to the site of injury, endothelial cells or their progenitors may be able to divide and form primitive blood vessels. Establishment of blood supply is fundamental for recovery of damaged tissues. PVMCs may have a pro-angiogenic effect via the secretion of bFGF, VEGF, placental growth factor (PlGF), and MCP-1 as well as angiogenic and anti-apoptotic factors such as IL-6, VEGF, and MCP-1, which inhibit the death of endothelial cells cultured under hypoxic conditions and promote the formation of capillary-like structures in in vitro assays. In some embodiments, isolated PVMCs may be able to contribute to angiogenesis by providing extracellular matrix components that serve as a substrate for endothelial cells. In some embodiments, isolated PVMCs may transition into pericytes and stabilize the newly formed vasculature.

In some embodiments, isolated PVMCs may secrete molecules that are powerful chemoattractants capable of recruiting various repair and helper cells into a regenerating tissue zone. In some embodiments, isolated PVMCs may secrete a variety of chemoattractant molecules, which include CCL2 (MCP-1), CCL3 (MIP-1α), CCL4 (MIP-1β), CCL5 (RANTES), CCL7 (MCP-3), CCL20 (MIP-3α), CCL26 (eotaxin-3), CX3CL1 (fractalkine), CXCL5 (ENA-78), CXCL11 (i-TAC), CXCL1 (GROα), CXCL12 (SDF-1), CXCL8 (IL-8), CXCL2 (GROβ), and CXCL10 (IP-10). Target cells for these chemoattractants may include monocytes, eosinophils, neutrophils, basophils, memory and naïve T-cells, B cells, NK cells, dendritic cells, and hematopoietic and endothelial progenitors. It is believed that chemokine expression of isolated PVMCs will be modified by exposure to other cell types, particularly immune cells.

In some embodiments, the isolated PVMCs are capable of expressing CD146, CD105, CD166, CD44, CD73, CD90, or a combination thereof. In some embodiments, the PVMCs are CD45 negative.

In some embodiments, isolated PVMCs can be utilized therapeutically. In some embodiments, isolated PVMCs can form part of an allogeneic infusate. In some embodiments, isolated PVMCs can be combined with isolated osteoblasts, whole marrow, unpurified, purified, or expanded PVMCs to form an infusate. In some embodiments, an infusate can also include a balanced salt solution comprising phosphate buffered saline, lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), or a combination thereof. In some embodiments, isolated PVMCs can be injected directly into a tissue. For example, a preparation of isolated PVMCs can be injected directly into the heart via a catheter. In some embodiments, isolated PVMCs can be encased in a scaffold prior to therapeutic administration. Examples of suitable scaffolds include preformed struts and crosslinking complexes activated by an enzyme or catalyst such that cross linking occurs in vivo.

In some embodiments, isolated PVMC preparations may be administered to the torso, head, or limbs of a human patient and may be capable of providing a regenerative microenvironment for bone regeneration. In some embodiments, the intrinsic secretory activity of isolated PVMCs may establish a regenerative microenvironment at sites of tissue injury. In some embodiments, isolated PVMCs may secrete bioactive factors that inhibit scarring, inhibit apoptosis, stimulate angiogenesis, and stimulate the mitosis and tissue-intrinsic stem or progenitor cells, and also secrete antibiotic proteins when bacteria may be present at the site of injury, for example, an open wound. The multifaceted effects of PVMCs can be referred to as “trophic activity”.

In some embodiments, the medicinal capabilities of the PVMCs are defined by the spectrum of molecules secreted by the PVMCs in a particular physiological environment as determined by specific, different anatomic locations.

In some embodiments, the PVMCs are obtained from a trabecular bone cavity of the bone. In some embodiments, PVMCs are obtained from the femoral head, the distal femur, or proximal tibia.

Some embodiments are directed to a method of stimulating bone regeneration comprising administering a composition comprising a therapeutically effective amount of PVMCs. In some embodiments, the composition further comprises bone marrow. In some embodiments, PVMC preparations are combined with osteoblasts.

In some embodiments, PVMC preparations are administered to the torso, head, or limbs of a human patient. In some embodiments, PVMCs are capable of providing a regenerative microenvironment.

In some embodiments, a method of treating a disease that affects cellular function comprises administering a composition comprising a therapeutically effective amount of PVMCs to a subject in need thereof. In some embodiments, the disease is ischemic heart disease, burns, stroke, inflammatory bowel disease, Crohn's disease, rheumatoid arthritis, lupus, amyotrophic lateral sclerosis, spinal cord damage, polytrauma, bone fractures, diabetes, or combinations thereof.

In some embodiments, the PVMCs are capable of secreting a site-dependent trophic factor. In some embodiments, a site-dependent effect of the trophic factor is selected from modulation of apoptosis; modulation of mitosis; modulation of angiogenesis; immunomodulation, modulation of scaring and fibrosis, or a combination thereof.

In some embodiments, the site dependent trophic factor is selected from prostaglandin E2 (PGE2), stromal-cell derived factor-1 (SDF-1), Vascular endothelial growth factor (VEGF), VEGF 165, interleukin-1β (IL-β), interleukin-6 (IL-6), interleukin-7 (IL-7), interleukin-8 (IL-8), interleukin-12 (IL-12), interleukin-16 (IL-16) vascular endothelial growth factor (VEGF), hepatocyte growth factor (HGF), transforming growth factor beta (TGF-β), basic fibroblast growth factor (bFGF), granulocyte-macrophage colony-stimulating factor (GM-CSF), insulin-like growth factor 1 (IGF-1), indoleamine 2,3-dioxygenase (IDO), interleukin-10 (IL-10), human leukocyte antigen G (HLA-G), leukemia inhibitory factor (LIF), class II major histocompatibility complex (MHC), eotaxin, granulocyte colony stimulating factor (G-csf), regulated upon activation, normal T-cell expressed and secreted (RANTES), IL-1 receptor antagonist (IL-1ra), tumor necrosis factor-α, TNF-α, tumor necrosis factor-β (TNF-β), epithelial neutrophil-activating protein 78 (ENA-78), eotaxin, monocyte chemoattractant protein 1 (MCP-1), monocyte chemoattractant protein 3 (MCP-3), macrophage inflammatory protein-1α (MIP-1α), macrophage inflammatory protein-3 α(MIP-3 α), macrophage inflammatory protein-1β (MIP-1 β), intercellular adhesion molecule-1 (ICAM-1), VCAM-1, granulocyte colony-stimulating factor (G-CSF), growth hormone, stem cell factor (SCF), thyroid-stimulating hormone (TSH), CD40 and CD40 ligand, placental growth factor (PlGF), eotaxin-3, fractalkine, epithelial neutrophil-activating protein 78 (ENA-78), Interferon-inducible T-cell alpha chemoattractant (i-TAC), growth regulated oncogene-alpha (GROα), growth regulated oncogene-beta (GROβ), Interferon-inducible protein-10 (IP-10), CD146, CD105, CD166, CD44, CD271, CD73, CD90, CD44, CD10, or a combination thereof.

In some embodiments, the PVMCs express CD271, CD73, CD90, or a combination thereof. In some embodiments, the PVMCs express CD146, CD105, CD44, CD10, or a combination thereof. In some embodiments, the perivascular cells do not express CD34, CD45, or a combination thereof.

In some embodiments, the PVMCs are obtained from a human donor. In some embodiments, PVMCs to be administered are autologous perivascular medicinal cells. In some embodiments, the PVMCs to be administered are allogeneic perivascular medicinal cells.

In some embodiments, PVMCs are obtained from human bone marrow, human tissue or undemineralized bone. In some embodiments, the human tissue is a capillary containing tissue selecting from fat, muscle, skin, placenta, umbilical cord tissue, vascular tissue, or a combination thereof.

In some embodiments, PVMCs are administered in conjunction with a pharmaceutically acceptable carrier. In some embodiments, the composition is administered by intravenous injection, intraperitoneal injection, direct tissue injection, or by direct application to the area as needed. In some embodiments, the composition may be directly applied to a burn or a wound. In some embodiments, the composition is administered intravenously. In some embodiments, the pharmaceutically acceptable carrier comprises standard infusion media. In some embodiments, standard infusion media comprises volume expanders, blood-based products, blood substitutes, buffer solutions, medications, nutrients, or combinations thereof. In some embodiments, volume expanders include but are not limited to D5W, 2/3D, 1/3S, half-normal saline, normal saline, Ringer's lactate, and D5NS. In some embodiments, blood-based products include, but are not limited to, whole blood, red blood cells, white blood cells, blood plasma, clotting factors and platelets. In some embodiments, blood substitutes include but are not limited to oxygen-carrying blood substitutes, hemoglobin-based oxygen carriers (HBOC) and perfluorocarbon-based oxygen carriers (PFBOC). In some embodiments, buffer solutions include but are not limited to lactated Ringer's solution and intravenous sodium bicarbonate. Nutrients include, but are not limited to, salts, glucose, amino acids, lipids and vitamins. Medications include, but are not limited to, those medications that would normally be administered via intravenous, intramuscular, subcutaneous, and intraperitoneal routes.

Some embodiments are directed to a method of modulating apoptosis comprising administering a composition comprising a therapeutically effective amount of PVMCs to a subject in need thereof. In some embodiments, the PVMCs are capable of modulating apoptosis in ischemic cells.

Some embodiments are directed to a method of modulating mitosis comprising administering a composition comprising a therapeutically effective amount of PVMCs to a subject in need thereof. In some embodiments, the PVMCs are capable of modulating mitosis in intrinsic tissue progenitors.

Some embodiments are directed to a method of modulating angiogenesis comprising administering a composition comprising a therapeutically effective amount of PVMCs to a subject in need thereof. In some embodiments, the PVMCs are capable of secreting a growth factor to modulate angiogenesis. In some embodiments, the PVMCs are localized to the perivascular tissue and stabilize newly formed blood vessels.

Some embodiments are directed to a method of reconstructing bone tissue comprising administering a composition comprising a therapeutically effective amount of PVMCs to a subject in need thereof.

Some embodiments are directed to a method of anchoring a metal device within a bone comprising administering a composition comprising a therapeutically effective amount of PVMCs to a subject in need thereof. In some embodiments, the metal device is anchored in a bone selected from cranial-facial bone, cranium, mandible, clavicle, scapula, sternum, ribs, humerus, ulna, radius, carpels, phalange, metacarpal, patella, fibula, femur, tibia, tarsal, metatarsal, sacrum, coxa or lumbar vertebrae.

In some embodiments, the PVMCs are capable of localizing to the perivascular tissue. In some embodiments, the PVMCs stabilize newly formed blood vessels. In some embodiments, the PVMCs secrete angiogenic molecules, vasculogenic molecules, or combinations thereof. In some embodiments, said angiogenic or vasculogenic molecules attract and multiply vascular endothelial cells. In some embodiments, administering the composition comprising a therapeutically effective amount of PVMCs results in recruitment and expansion of endothelial cells.

Some embodiments are directed to a method of modulating bone formation comprising administering a composition comprising a therapeutically effective amount of PVMCs to a subject in need thereof. In some embodiments, the PVMCs have the capability to form osteoblasts.

Some embodiments are directed to a method of immunomodulation comprising administering a composition comprising a therapeutically effective amount of PVMCs to a subject in need thereof. In some embodiments, the immunomodulation is mediated through at least one of a cytokine, a growth factor, or a combination thereof. In some embodiments, the immunomodulation is in the lymph or lymphatic system.

Some embodiments are directed to a composition comprising PVMCs and scaffold material. In some embodiments, the PVMCs have been cryopreserved and subsequently thawed. In some embodiments, the scaffold material comprises bone chips, ceramic-based bone graft substitutes, calcium phosphate ceramics, calcium sulfate ceramics, bioglass, polymer-based bone graft substitutes, degradable and nondegradable polymers, processed allograft bone material, mineralized processed allograft, demineralized processed allograft, collagen sponges, or combinations thereof.

In some embodiments, the PVMC preparations described in the present disclosure can be administered to a human subject to treat disease that affects cellular function. The PVMCs may be used alone or in combination with other therapeutic agents. When used alone, PVMC preparations may be injected intravenously or at the site of injury. Therapeutic regimens may be composed of multiple injections defined time course or with a single injection. Cell-based therapy such as the embodiments described in the present disclosure may have the advantage of exerting multiple therapeutic effects at various sites and times within the lesion as the cells respond to a particular pathological micro-environment.

PVMC preparations for therapeutic administration may include autologous bone grafts prepared from a first portion of bone supplemented with a population of concentrated bone or umbilical cord-derived PVMCs. In some embodiments, the concentrated bone or umbilical cord-derived PVMCs may be prepared by extracting and concentrating the PVMCs from a second portion of the same autologous bone tissue from the subject so as to make the enhanced, autologous bone graft.

In some embodiments, PVMC preparations can form part of an allogeneic infusate. Bone or umbilical cord-derived PVMCs will be combined with isolated osteoblasts, whole marrow, unpurified, purified, or expanded PVMCs to form an infusate. In some embodiments, the infusate can also include a balanced salt solution such as, without limitation, phosphate buffered saline, lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), or a combination thereof.

In some embodiments, the PVMC preparation may be injected directly into a tissue. For example, a preparation of bone derived PVMCs may be injected directly into the heart via a catheter. In some embodiments, the method of treating a disease may comprise encasing a PVMC preparation in a scaffold. PVMC preparations may be encased in a scaffold prior to therapeutic administration. Examples of suitable scaffolds include preformed struts and crosslinking complexes activated by an enzyme or catalyst such that cross linking occurs in vivo.

In some embodiments, a preparation of PVMC may be combined with bone chips to form a paste that may be applied directly to a wound site. The paste may be able to provide a regenerative microenvironment. In some embodiments, the paste may have anti-apoptotic properties.

PVMC preparations may be administered with a wide variety of additional elements including, but not limited to, synthetic bone substitutes, wherein the synthetic bone substitutes comprise a calcium phosphate-based bone substitute, calcium apatite, β-tricalcium phosphate, natural and synthetic polymers, ceramics, Allogro, Opteform, Grafton, OrthoBlast, calcium phosphate, calcium sulfate, bioglass, OsteoGraf, Norian SRS, ProOsteon, Osteoset, polymer-based bone graft substitutes, degradable and nondegradable polymers, Cortoss, open porosity polylactic acid polymer, Immix, or combinations thereof; mineralized processed allograft, demineralized processed allograft, or a combination thereof; collagen sponge, BMP-2-containing collagen sponge, BMP-7-containing collagen sponge, BMP-2 and BMP-7 containing sponge, or combinations thereof; PDGF-BB; calcium phosphate-based bone substitute combined with isolated osteoblasts, whole marrow, unpurified, purified, or expanded PVMCs, or combinations thereof; bone substitutes such as hydroxyapatite, calcium apatite, β-tricalcium phosphate, natural and synthetic polymers, ceramics, or other bone substitute materials, or combinations thereof, in addition to comprising isolated osteoblasts, whole marrow, unpurified, purified or expanded PVMCs; discarded knee or hip bone/marrow obtained during route arthroplasty. In some embodiments, the PVMCs may be used alone or in combination with bone or other scaffolds such as autologous bone grafts.

### Methods for Storing Isolated PVMCs and PVMC Preparations

In some embodiments, isolated PVMC preparations, isolated PVMCs, isolated PVMCs derived from bone or bone tissue, isolated PVMCs derived from umbilical cord blood vessels, or a combination thereof can be stored for future use. Storage of isolated PVMC preparations, isolated PVMCs, isolated PVMCs derived from bone or bone tissue, isolated PVMCs derived from umbilical cord blood vessels, or a combination thereof, can be achieved by cryogenic preservation at temperatures ranging from −20° C. to −250° C. In some embodiments, isolated PVMC preparations, isolated PVMCs, isolated PVMCs derived from bone or bone tissue, isolated PVMCs derived from umbilical cord blood vessels, or a combination thereof may be mixed with plasma-lite and dimethyl sulfoxide and then stored in liquid nitrogen or liquid nitrogen vapor. In some embodiments, isolated PVMC preparations, isolated PVMCs, isolated PVMCs derived from bone or bone tissue, isolated PVMCs derived from umbilical cord blood vessels, or a combination thereof may be mixed with about 0.5 M ethylene glycol, about 1.0 M propylene glycol and about 1.5 M dimethyl sulfoxide in the presence of culture medium and then stored in liquid nitrogen or liquid nitrogen vapor. In some embodiments, isolated PVMC preparations, isolated PVMCs, isolated PVMCs derived from bone or bone tissue, isolated PVMCs derived from umbilical cord blood vessels, or a combination thereof may be mixed with culture medium and 10% dimethyl sulfoxide and then stored in liquid nitrogen or liquid nitrogen vapor. In some embodiments, the process of freezing an isolated PVMC preparations, isolated PVMCs, isolated PVMCs derived from bone or bone tissue, isolated PVMCs derived from umbilical cord blood vessels, or a combination thereof is achieved by flash freezing. As described herein, flash freezing includes a process of immersing a PVMC preparation into liquid nitrogen resulting in rapid freezing of the isolated PVMC preparations, isolated PVMCs, isolated PVMCs derived from bone or bone tissue, isolated PVMCs derived from umbilical cord blood vessels, or a combination thereof. In some embodiments, the process of freezing an isolated PVMC preparations, isolated PVMCs, isolated PVMCs derived from bone or bone tissue, isolated PVMCs derived from umbilical cord blood vessels, or a combination thereof, is achieved by gradual lowering of the temperature of the PVMC preparation by immersion of the preparation to a liquid nitrogen vapor. In some embodiments, isolated PVMC preparations, isolated PVMCs, isolated PVMCs derived from bone or bone tissue, isolated PVMCs derived from umbilical cord blood vessels, or a combination thereof are placed in a freezing chamber coupled to a temperature probe, wherein the temperature is lowered by a computer controlled protocol to allow for a gradual descent to a desired freezing temperature. In a further embodiment, isolated PVMC preparations, isolated PVMCs, isolated PVMCs derived from bone or bone tissue, isolated PVMCs derived from umbilical cord blood vessels, or a combination thereof can be packaged in cryovials containing predetermined amounts of PVMCs and cooled at approximately −1° C./minute using a dump-freeze method consisting of suspension of vials in an isopropanol bath within a −85° C. mechanical freezer for 24 hours, followed by plunge into liquid nitrogen for storage at −196° C. In some embodiments, isolated PVMC preparations, isolated PVMCs, isolated PVMCs derived from bone or bone tissue, isolated PVMCs derived from umbilical cord blood vessels, or a combination thereof, can be retrieved from storage and immediately thawed in a 37° C. water bath followed by careful washing in a sterile medium to remove the cryoprotectant by slow dilution with complete medium over 10 minutes followed by centrifugation at 500×g for 5 minutes, aspiration of supernatant and resuspension in fresh complete medium.

In some embodiments, cryogenically frozen isolated PVMC preparations, isolated PVMCs, isolated PVMCs derived from bone or bone tissue, isolated PVMCs derived from umbilical cord blood vessels, or a combination thereof, may be thawed by placing the preparation in a pre-heated sterile water bath at a temperature of about 35° C. to 40° C. Once immersed in the water bath, the isolated PVMC preparations, isolated PVMCs, isolated PVMCs derived from bone or bone tissue, isolated PVMCs derived from umbilical cord blood vessels, or a combination thereof, are mixed until thawed. The isolated PVMC preparations, isolated PVMCs, isolated PVMCs derived from bone or bone tissue, isolated PVMCs derived from umbilical cord blood vessels, or a combination thereof, is then suitable for expansion in cell culture or testing for PVMC viability. In some embodiments, isolated PVMC preparations, isolated PVMCs, isolated PVMCs derived from bone or bone tissue, isolated PVMCs derived from umbilical cord blood vessels, or a combination thereof may be thawed by immersing the preparation in liquid nitrogen vapor for about 30 to 45 minutes. The PVMC preparation is subsequently maintained in dry ice until it is needed for expansion in cell culture, testing or therapeutic use.

This invention and embodiments illustrating the method and materials used may be further understood by reference to the following non-limiting examples.

### EXAMPLES

### Example 1

### Isolation of PVMCs from an Umbilical Cord Blood Vessel

PVMCs will be isolated from a vein or artery of an umbilical cord blood vessel via a multi-step process. 4-5 cm long portions of umbilical cord blood vessel will be isolated and immersed in Tyrode's solution containing antibiotics (300 units/mL penicillin, 300 μg/mL streptomycin, 150 μg/mL gentamicin, and 1 μg/mL fungizone or alternatively, immersed in 80% α-MEM containing 20% antibiotics (167 units/mL penicillinG, 50 μg/mL gentamicin, 0.3 μg/mL amphotericin). PVMCs should be isolated from umbilical cord blood vessels within 6 to 12 hours from obtaining umbilical cord blood vessel tissue ex utero.

In a first step, the umbilical cord blood vessel will be canulated and washed with Tyrode's solution containing 100 units/mL heparin and the wash solution is discarded.

In a second step, the distal end of the umbilical cord blood vessel will be clamped and the canulated umbilical cord blood vessel or vein is filled with α-MEM containing 1 mg/mL collagenase (type IV) or alternatively an optimized enzyme mix. In an alternative step, collagenase (type IV) is replaced with a metalloproteinase.

In a third step, the proximal end of the umbilical cord blood vessel will be clamped and the umbilical containing collagenase or the optimized enzyme mixture is incubated for 20-30 minutes at 37° C. The umbilical cord blood vessel is then unclamped and the α-MEM containing 1 mg/mL collagenase (type IV) or alternatively an optimized enzyme mix is drained from the vein or artery.

In a fourth step, the umbilical cord blood vessel will be re-clamped and washed with Tyrode's solution followed by gentle massaging of the umbilical cord blood vessel. The resulting Tyrode's solution contains a suspension of endothelial and subendothelial cells. The Tyrode's' cell suspension is collected and the cells are washed and subsequently cultured in DMEM supplemented with 10% FBS, 2 mM L-glutamine, 100 units/mL penicillin and 100 units/mL streptomycin at 37° C. with 5% CO₂ for 3 days. This process will yield a population of cells largely comprising endothelial cells with a small PVMC population.

In a fifth step, the second through fourth steps will be repeated to yield a second Tyrode's cell suspension that can be cultured in DMEM supplemented with 10% FBS, 2 mM L-glutamine, 100 units/mL penicillin and 100 units/mL streptomycin at 37° C. with 5% CO₂ for 3 days. This second cell suspension is expected to be enriched for PVMCs.

### Example 2

### Isolation of PVMCs from an Umbilical Cord Blood Vessel

PVMCs will be isolated from a vein or artery of an umbilical cord blood vessel via a multi-step process including two separate enzyme incubations. In a first step, 4-5 cm long portions of umbilical cord blood vessel will be isolated and immersed in Tyrode's solution containing antibiotics (300 units/mL penicillin, 300 μg/mL streptomycin, 150 μg/mL gentamicin, and 1 μg/mL fungizone or alternatively, immersed in 80% α-MEM containing 20% antibiotics (167 units/mL penicillinG, 50 μg/mL gentamicin, 0.3 μg/mL amphotericin). PVMCs should be isolated from umbilical cord blood vessels within 6 to 12 hours from obtaining umbilical cord blood vessel tissue ex utero.

In a second step, the portion of umbilical cord will be attached in a vertical position to a ring stand and the top and bottom section of the umbilical cord are clamped. This is followed by cannulation of the top and bottom sections of the blood vessel and inserting a 3-way port into the top and bottom sections of the blood vessel of the suspended umbilical cord blood vessel, wherein the 3-way port in the top section is capable of allowing delivery of a medium into the blood vessel. The 3-way port allows for insertion of a medium, movement of air in and out of the blood vessel, and emptying of medium contained in the blood vessel.

In a third step, the umbilical cord blood vessel will then be washed with Tyrode's solution containing 100 units/mL heparin and the wash solution is discarded. The Tyrode's is injected into the 3-way port inserted into the bottom section of the umbilical cord blood vessel until the vessel is full of solution. The Tyrode's solution is then emptied through the same 3-way port.

In a fourth step, the umbilical cord blood vessel will be filled with α-MEM containing 1 mg/mL collagenase (type IV) or alternatively an optimized enzyme mix. In an alternative step, collagenase (type IV) is replaced with a metalloproteinase. The α-MEM containing 1 mg/mL collagenase (type IV) or alternatively an optimized enzyme mix is injected into the 3-way port inserted into the bottom section of the umbilical cord blood vessel until the vessel is full of medium. The α-MEM containing 1 mg/mL collagenase (type IV) or alternatively an optimized enzyme mix is then emptied through the same 3-way port.

In a fifth step, the umbilical cord blood vessel containing α-MEM containing 1 mg/mL collagenase (type IV) or alternatively an optimized enzyme mix is incubated for 20-30 minutes at 37° C. The umbilical cord blood vessel is emptied through the 3-way port in the bottom section of the umbilical cord blood vessel and discarded.

In a sixth step, the second through fourth steps will be repeated to yield a second Tyrode's cell suspension that can be cultured in DMEM supplemented with 10% FBS, 2 mM L-glutamine, 100 units/mL penicillin and 100 units/mL streptomycin at 37° C. with 5% CO₂ for 3 days. This second cell suspension is expected to be enriched for PVMCs.

During the process of isolating PVMCs from the umbilical cord blood vessel, it is possible to cryogenically preserve the umbilical cord. Cryogenic preservation is envisioned after immersion in Tyrode's solution in the first step of the isolating process. Cryogenic preservation of the umbilical cord is also envisioned following the third step of isolating process, that is, after washing the umbilical cord blood vessel with Tyrode's solution to flush an residual blood remaining in the vessel. Once the blood vessel has been washed as described in the second step, it may be filled with a suitable cryoprotection solution such as glycerol, ethylene glycol, propylene glycol, or another glycol containing dimethyl sulfoxide. The umbilical cord section is then snap frozen to avoid the formation of ice crystals and damage to the tissue and cells contained within the blood vessels. The umbilical cord section can also be frozen in a stepwise manner via an initial freezing phase at −70° C. for about 1 to 24 hours followed by long term cryogenic preservation at −196° C. in liquid nitrogen. Cryogenically preserved samples can be thawed for completion of the isolating protocol. To maintain tissue and cell viability, cryogenically preserved samples should be rapidly thawed.

### Example 3

### Culturing of PVMCs

PVMCs isolated from an umbilical cord blood vessel may be cultured to selectively expand a population of concentrated umbilical cord blood vessel-derived PVMCs. One or more cell suspensions isolated from an umbilical cord blood vessel may be diluted with DMEM supplemented with 10% fetal bovine serum (FBS). The DMEM mixture may be vigorously vortexed to mechanically disperse the cells followed by centrifugation at 480×g for 5 minutes in a bench top centrifuge after which the supernatant is removed. The remaining cell pellet will be fractionated to collect nucleated cells using Percoll™ (density 1.03-1.12 g/mL) followed by a second round of centrifugation at 480×g for 15 minutes without breaking to ensure an intact Percoll™ gradient. The top fraction of the gradient is then transferred to a new tube and supplemented with DMEM followed by centrifugation at 480×g for 15 minutes. After centrifugation, the supernatant is removed without disturbing the pellet. The pellet is then resuspended in DMEM and washed several times by centrifugation using DMEM. The resulting PVMC cell suspension is then ready for expansion or concentration.

### Example 4

### Isolation of PVMCs from the Femoral Head of the Femur

Discarded bone fragments or the femoral head from hip replacement surgery can be used as bone chips as well as a source of PVMCs. PVMCs can be isolated by first extracting a cell suspension from the bone by enzymatic digestion, and mechanical force; and second, by concentrating the extracted PVMCs and selectively expanding a PVMC population.

In order to extract a cell suspension from the femoral head of the femur, the bone may be ground. The bone is first cleaned of extraneous soft tissue, and milled to achieve cortical/cancellous chips in the form of irregularly shaped polyhedra with an edge dimension up to 5 mm. The bone chips may comprise bone marrow. In some cases, bone chips will be obtained from the femoral head and the cleaning process is omitted to retain bone marrow tissue in the preparation. In addition to mechanically breaking down bone, enzymatic digestion of the bone fragments can be achieved by subjecting the bone to a series of treatments with the following enzymes: collagenase, proteases, GAGases, and metalloproteases, clostripain (a cysteine protease from C. histolyticum), serine proteases, alkaline proteases, cysteine proteases, or combinations thereof. Without wishing to be bound by theory, it is believed that enzymatic digestion specifically cleaves bonds joining the PVMCs to the basement membrane of small blood vessels within the bone fragments. In some embodiments, the enzymatic digestion of bone fragments can be performed subsequently to mechanical breakdown of bone as well as with intact fragments of bone.

Following enzymatic digestion, the cell suspensions obtained from bone fragments may be cleaned and collected by density gradient centrifugation using a Smartprep2™ centrifuge at 2,500 rpm for 3 minutes followed by 2,300 rpm for 9 minutes. Alternatively, cells can be recovered by gravity, with particles collecting at the bottom of a collection tube.

PVMC cell suspensions may be then concentrated by the use of magnetic beads comprising antibodies with affinity to cell surface antigens on the PVMC. Concentration of PVMC may be performed upon an expanded cell population. The concentration of a PVMC population may be performed without prior expansion of the cell population. PVMC preparations may be administered in an autologous fashion. Absolute cell purity may not be required. Without wishing to be bound by theory, it is believed that impurities in endothelial cells or monocytes may be beneficial to bone forming events.

In some embodiments, the concentrate may be subsequently mixed with porous hydroxyapatite granules (Orthoss®; 97 m²/g, total porosity 60%, intercrystalline spaces crystal size 10-60 mm, Ca/P 2.03) or applied onto a porcine or bovine collagen sponge (100 mg gelatin, resorption period in vivo 2-3 weeks). The concentrate may be encased in an autologous fibrin clot.

These preparations may be suitable for direct use in orthopedic, dental and craniofacial reconstruction applications. Isolated PVMCs can be stored for future use by cryogenic preservation at −195° C.

### Example 5

### Administration of PVMCs to Create a Regenerative Microenvironment for Bone Regeneration

PVMC preparations such as those from Example 1 will be administered to a human subject to promote the regeneration of bone following an injury. These cells may be used alone or in combination with bone or other scaffolds such as autologous bone grafts. When used alone, PVMC preparations can be injected intravenously or at the site of injury. Therapeutic regimens can be composed of multiple injections defined time course or with a single injection. Therapeutic regimens can also be infused via intravenous infusion into the vein of a patient by injecting a PVMC preparation via an injection port into a bag of saline connected to a catheter that has been previously inserted into the vein of a patient. A cell based-therapy such as the embodiments described in this application may have the advantage of exerting multiple therapeutic effects at various sites and times within the lesion as the cells respond to a particular pathological micro-environment.

PVMC preparations such as those in example 1 can form part of an allogeneic infusate. Bone-derived PVMCs can be combined with isolated osteoblasts, whole marrow, unpurified, purified, or expanded PVMCs to form an infusate for intravenous administration. An infusate can also include a balanced salt solution comprising phosphate buffered saline, lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), or a combination thereof. Alternatively, bone-derived PVMCs can be injected directly into a tissue. For example, a preparation of bone-derived PVMCs can be injected directly into the heart via a catheter. Furthermore, bone-derived PVMCs can be encased in a scaffold prior to therapeutic administration directly to the site of injury. For example bone derived PVMCs combined with bone chips to form a paste can be placed directly onto an injured vertebrae or another bone that has been surgically exposed and prepared. Other examples of suitable scaffolds include preformed struts and crosslinking complexes activated by an enzyme or catalyst such that cross linking occurs in vivo.

### Example 6

### Use of PVMC and Bone Chip Composites in Tooth Extraction and Replacement with a Dental Implant

A dental implant is a “root” device, usually made of titanium, used in dentistry to support restorations that resemble a tooth or group of teeth to replace missing teeth. Virtually all dental implants placed today are root-form endosseous implants, i.e., they appear similar to an actual tooth root and thus possess a “root-form” and are placed within the bone cavity where the dental root was previously located prior to a tooth extraction. The bone of the jaw accepts and osseointegrates with the titanium post. Osseointegration refers to the fusion of the implant surface with the surrounding bone.

The placement of an osseointegrated implant requires a preparation into the bone using either hand osteotomes or precision drills with highly regulated speed to prevent burning or pressure necrosis of the bone. After a variable amount of time to allow the bone to grow on to the surface of the implant (osseointegration), a crown or crowns will be placed on the implant. The amount of time required to place an implant may vary depending on the experience of the practitioner, the quality and quantity of the bone and the difficulty of the individual situation.

PVMC and bone chip composites can be utilized in plugging the cavity remaining following tooth extraction or surrounding the implant so as to promote osseointegration. The PVMC and bone chip composition preparation can also be used to fill a tooth extraction site, following tooth extraction. The PVMC and bone chip preparation may provide the necessary regenerative microenvironment to stimulate bone formation within the tooth extraction site. The PVMC and bone chip composite preparation is inserted into a prepared tooth extraction site. Following insertion of the PVMC and bone chip composite preparation into the prepared tooth extraction site, the dental implant is inserted into the filled tooth extraction site onto which a prosthetic tooth will be attached. The resulting regenerative microenvironment created by the presence of the PVMC and bone chip composite preparation will result in bone formation around the metal post.

### Example 7

### Preparation of Partially Demineralized Bone

Bone from a femur head will be ground into bone chips, milled and placed into a sieve to isolate milled bone having a size of about 800 microns. The bone chips may contain bone marrow. The milled bone may have a combination of bone chips and bone powder. The milled bone material will be placed in a mixing container and cleaned with a 5:1 ratio of 3% Hydrogen Peroxide and will be stirred for 15 minutes, removed and rinsed with a minimum of 3000 mL of sterile water. The rinsed bone material will be placed back into the cleaned mixing container and at least 1000 ml of 70% EtOH will be added and the solution will be mixed for 30 minutes. The bone material will be transferred into a No. 70 sieve and an open vacuum is applied to the bottom of the sieve and the bone powder is dried for 20 minutes. The dried bone material will be weighed. The bone weight in grams will be compared to a chart which determines the acid volume to be applied, in which approximately 1 gram of bone would require approximately 16 mL of acid. The bone material will be mixed with 0.6 M hydrochloric acid for about 7 hours to partially remove mineral content. The bone material may be mixed with hydrochloric acid for a longer time (up to 24 hours) to remove more mineral content.

### Example 8

### Obtaining Bone Chips from Bone

Obtaining bone chips from bone is a multistep process. Suitable bone originates from human bones comprising ilium crest, femur, patella, tibia, humerus, clavicle, ribs, or scapula, or combinations thereof. In some embodiments, this tissue may be obtained as discarded tissue following surgical operations on patients and prepared for autologous use. In some embodiments, bone can originate from the proximate and distal regions of the femur, ilium crest, patella, tibia, humerus, clavicle, ribs, or scapula.

In a first step, a suitable bone mill such as a Noviomagus Bone Mill is first assembled according to the manufacturer's instructions on a flat surface, using a “fine milling” for small size bone particles. A suitable bowl or collection receptacle is then placed under the milling drum outlet.

In a second step, a fragment of bone is obtained which can be frozen prior to or subsequent to the grinding process or fresh. To prepare the isolated bone for milling, excess tissue and articular cartilage is trimmed with a bone cutter.

In a third step, the fragment of bone is placed into the housing of the mill. When using bone from the femoral head, the head is usually cut in half and head halves are milled separately with the trabecular bone surface facing opposite to the direction of the rotation of the mill. Clockwise rotation of the mill handle while applying downward pressure on the mill's push block results in milling of bone fragments to bone chips. The bone chips are collected in the bowl or collection receptacle. Excess bone chips are also retrieved from within the milling drum and housing where they may be retained during the milling process with a spatula.

Bone chips may comprise both bone and marrow tissue. Alternatively, a bone chip may comprise compact bone, bone marrow, tissue from the medullary canal, cancellous tissue, or combinations thereof. Bone chips formed by this method vary in size depending on the origin of the bone. For example, bone chips from a proximal tibia may be milled to form particles ranging in size from about 3.6 mm to about 8.0 mm; bone chips, from a distal femur, may be milled to form particles ranging in size from about 2.9 mm to about 7.1 mm; bone chips, from a femoral head, may be milled to form particles ranging in size from about 2.2 mm to about 3.4 mm. The size of a milled bone chip may be measured around its largest axis.

Bone can subsequently be cryogenically preserved or used immediately for therapeutic administration.

### Example 9

### Preparation of Partially Demineralized Bone Chips

Bone from a femur head will be ground into bone chips measuring about 3.0 mm around its largest axis. The bone chips may contain bone marrow. The bone chips will be placed in a mixing container and cleaned with a 5:1 ratio of 3% hydrogen peroxide and will be stirred for 15 minutes, removed and rinsed with a minimum of 3000 mL of sterile water. The rinsed bone material will be placed back into the cleaned mixing container and at least 1000 mL of 70% EtOH will be added and the solution will be mixed for 30 minutes. The bone material will be transferred into a sieve and an open vacuum is applied to the bottom of the sieve and the bone chips are dried for 20 minutes. The dried bone material will be weighed. The bone weight in grams will be compared to a chart which determines the acid volume to be applied, in which approximately 1 gram of bone would require approximately 16 mL of acid. The bone chips will be mixed with 0.6 M Hydrochloric acid for about 7 hours to partially remove mineral content. The bone chips may be mixed with Hydrochloric acid for a longer time (up to 24 hours) to remove more mineral content.

### Example 10

### Culturing of PVMCs Directly from Bone Chips

PVMCs may be cultured directly from bone chips to selectively expand a population of concentrated bone-derived PVMCs. A milled bone sample containing bone tissue is diluted with DMEM supplemented with 10% fetal bovine serum (FBS). The bone-DMEM mixture may be vigorously vortexed to mechanically disperse the tissue and separate it from the bone followed by centrifugation at 480×g for 5 minutes in a bench top centrifuge after which the supernatant is removed. The remaining cell pellet will be fractionated to collect nucleated cells using Percoll™ (density 1.03-1.12 g/mL) followed by a second round of centrifugation at 480×g for fifteen minutes without breaking to ensure an intact Percoll™ gradient. The top fraction of the gradient is then transferred to a new tube and supplemented with DMEM followed by centrifugation at 480×g for 15 minutes. After centrifugation, the supernatant is removed without disturbing the pellet. The pellet is then resuspended in DMEM and washed several times by centrifugation using DMEM. The resulting PVMC cell suspension is then ready for expansion or concentration.

Although the present invention has been described in considerable detail with reference to certain preferred embodiments thereof, other versions are possible. Therefore, the spirit and scope of the appended claims should not be limited to the description and the preferred versions contained within this specification.

### Example 11

### Administration of PVMCs to Create a Regenerative Microenvironment for Bone Regeneration

PVMC preparations will be administered to a human subject to promote the regeneration of bone following an injury. The PVMC preparations include bone marrow. The PVMC preparations will be combined with a bone powder and can be injected intravenously or at the site of injury. Therapeutic regimens can be composed of multiple injections defined time course or with a single injection. Therapeutic regimens can also be infused via intravenous infusion into the vein of a patient by injected a PVMC preparation via an injection port into a bag of saline connected to a catheter that has been previously inserted into the vein of a patient.

### Example 12

### Administration of PVMCs in an Infusate for Bone Regeneration

PVMC preparations will be administered to a subject in need thereof as an allogeneic infusate. Bone or umbilical cord-derived PVMCs will be combined with isolated osteoblasts, whole marrow, unpurified, purified or expanded PVMCs to form an infusate for intravenous administration. The infusate can also include a balanced salt solution such as, without limitation, phosphate buffered saline, lactated Ringer's solution, acetated Ringer's solution, TRIS-buffered saline (TBS), Hank's balanced salt solution (HBSS), Earle's balanced salt solution (EBSS), Standard saline citrate (SSC), HEPES-buffered saline (HBS), Gey's balanced salt solution (GBSS), or a combination thereof. Alternatively, bone or umbilical cord-derived PVMCs will be injected directly into a tissue. For example, a preparation of bone derived PVMCs will be injected directly into the heart via a catheter.

### Example 13

### Direct Administration of PVMCs for Bone Regeneration

Bone or umbilical cord-derived PVMCs will be encased in a scaffold prior to therapeutic administration directly to the site of injury. Bone or umbilical cord-derived PVMCs combined with bone chips to form a paste can be placed directly onto an injured vertebrae or another bone that has been surgically exposed and prepared. Other examples of suitable scaffolds include, without limitation, pre-formed struts and crosslinking complexes activated by an enzyme or catalyst such that cross linking occurs in vivo.

Although the present invention has been described in considerable detail with reference to certain preferred embodiments thereof, other versions are possible. Therefore the spirit and scope of the appended claims should not be limited to the description and the preferred versions contained within this specification.

1.-74. (canceled)

75. A composition comprising pure, substantially pure, or impure isolated PVMCs, wherein the PVMCs have medicinal capabilities.

76. The composition of claim 75, wherein the isolated PVMCs may be capable of expressing CD146, CD105, CD166, CD44, CD73, CD90, or a combination thereof.

77. The composition of claim 75, wherein the isolated PVMCs are isolated from bone.

78. The composition of claim 77, wherein bone comprises a bone chip, a trabecular bone cavity, bone marrow, bone cavity lavage, or a combination thereof.

79. The composition of claim 75, wherein the medicinal capabilities of the isolated PVMC are defined by the spectrum of molecules secreted by the PVMC.

80. The composition of claim 79, wherein the spectrum of molecules secreted by the isolated PVMC is site dependent.

81. The method of claim 75, wherein the pure, substantially pure or impure isolated PVMCs further comprise bone chips, fully demineralized bone chips, or partially demineralized bone chips.

82. A method for isolating PVMCs from bone the method comprising: (i) providing a sample of bone tissue from a subject; (ii) extracting the PVMCs from the bone; and (iii) concentrating the extracted PVMCs.

83. The method claim 82 further comprising grinding the bone tissue.

84. The method of claim 82, wherein extracting the PVMCs comprises: (i) extracting a cell suspension from the bone tissue by enzymatic digestion, mechanical force, or a combination thereof; and (ii) separating a population of PVMCs from the cell suspension by buoyant density sedimentation, filtration, centrifugation, or a combination thereof.

85. The method of claim 84, wherein the enzymatic digestion uses one or more enzymes that cleave the attachment of a PVMC from a basement membrane of a small blood vessel.

86. The method of claim 85, wherein enzymatic digestion may be achieved by using one or more enzymes selected from collagenase, neutral or acidic proteases, GAGases, or metalloproteases, clostripain, serine proteases, alkaline proteases, cysteine proteases, or combinations thereof.

87. A method for making an enhanced, autologous bone graft comprising: a.) extracting a cell suspension from a first portion of bone tissue from a subject with an enzyme, mechanical force, or a combination thereof; b.) concentrating the cells in the cell suspension by buoyant density sedimentation, filtration or centrifugation to obtain a population of concentrated bone-derived PVMCs; and c.) supplementing a second portion of bone tissue to be used as a bone graft from the subject with the population of concentrated bone-derived PVMCs, so as to make the enhanced, autologous bone graft.

88. The method of claim 87, further comprising the addition of mineralized processed allograft, minimally demineralized processed allograft, partially demineralized processed allograft, demineralized processed allograft, or a combination thereof.

89. The method of claim 87, further comprising the step of supplementing an enhanced, autologous bone graft with fresh autologous bone marrow, processed autologous bone marrow, frozen autologous bone marrow, or combinations thereof.

90. A method of producing bone chips comprising passing a bone fragment through a grinder or bone mill.

91. The method of claim 90, wherein a bone chip comprises compact bone, bone marrow, tissue from the medullary canal, cancellous tissue, or combinations thereof.

92. The method of claim 90, wherein the bone fragment is cryogenically frozen.

93. The method of claim 90, wherein a bone fragment is milled to form a bone chip ranging in size from 3.6 mm to 8.0 mm.

94. The method of claim 90, further comprising therapeutic administration to a patient in need thereof.
